SEQUENCE LISTING (7 sequences)

SEQ ID NO: 1 - protein, 135 aa, Hepatitis C virus
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

SEQ ID NO: 2 - protein, 46 aa, HCV
YEVRNVSGMYHVTNDCSNSSIVYEAADMIMHTPGCVPCVRENNSSR

SEQ ID NO: 3 - protein, 20 aa, Artificial Sequence
  Description of Artificial Sequence igp 263
NDCPNSSIVYEAHDAILHTP

SEQ ID NO: 4 - protein, 22 aa, Artificial Sequence
  Description of Artificial SequenceIGP 436
GGSNSSIVYEAHDAILHTPGCV

SEQ ID NO: 5 - protein, 8 aa, Artificial Sequence
  UNSURE (5): X=ANY BASE
YEVRXVSG

SEQ ID NO: 6 - protein, 11 aa, Artificial Sequence
  UNSURE (1)..(2): x=ANY BASE
XXVALTPTLAA

SEQ ID NO: 7 - protein, 4 aa, Artificial Sequence
  UNSURE (2)..(3): X=ANY BASE
CXXC

(19) United States
(12) Patent Application Publication
(10) Pub. No.: US 2004/0185061 A1
(43) Pub. Date: Sep. 23, 2004

(54) Redox reversible HCV proteins with native-like conformation

(75) Inventors: Alfons Bosman, Opwijk (BE); Erik Depla, Destelbergen (BE); Geert Maertens, Brugge (BE)

Correspondence Address: NIXON & VANDERHYE, PC, 1100 N GLEBE ROAD, 8TH FLOOR, ARLINGTON, VA, 22201-4714, US

(73) Assignee: Innogenetics N.V., Ghent (BE)

(21) Appl. No.: 10/825,219

(22) Filed: Apr. 16, 2004

Related U.S. Application Data
(63) Continuation of application No. 09/795,289, filed on Dec. 7, 1999.
(63) Continuation-in-part of application No. 09/973,025, filed on Oct. 10, 2001.
Application No. 09/973,025 is a continuation of application No. 08/928,017, filed on Sep. 11, 1997, now abandoned.
Application No. 08/928,017 is a division of application No. 08/612,973, filed on Mar. 11, 1996, now Pat. No. 6,150,134.
Application No. 08/612,973 is a 371 of international application No. PCT/EP95/03031, filed on Jul. 31, 1995.

(30) Foreign Application Priority Data
Oct. 27, 1999 (EP) 99870225.2
Jul. 29, 1994 (EP) 94870132.1

(51) Int. Cl. C12Q 1/70; A61K 39/29; C07K 14/02

(52) U.S. Cl. 424/189.1; 435/5; 530/350

(57) ABSTRACT

The present invention relates to HCV proteins in which cysteine residues are reversibly protected during purification. Eventually, this purification procedure results in HCV proteins with biological activity and a native-like protein conformation, which present corresponding epitopes. The present invention pertains also to drug screening methods using these HCV proteins, and diagnostic and therapeutic applications, such as vaccines and drugs.

### FIELD OF THE INVENTION

The present invention relates to HCV proteins in which cysteine residues are reversibly protected during purification. Eventually, this purification procedure results in HCV proteins with biological activity and a native-like protein conformation, which present corresponding epitopes. The present invention pertains also to drug screening methods using these HCV proteins, and diagnostic and therapeutic applications, such as vaccines and drugs.

### BACKGROUND OF THE INVENTION

Hepatitis C virus (HCV) infection is a major health problem in both developed and developing countries. It is estimated that about 1 to 5% of the world population is affected by the virus. HCV infection appears to be the most important cause of transfusion-associated hepatitis and frequently progresses to chronic liver damage. Moreover, there is evidence implicating HCV in induction of hepatocellular carcinoma. Consequently, the demand for reliable diagnostic methods and effective therapeutic agents is high. Also sensitive and specific screening methods of HCV-contaminated blood-products and improved methods to culture HCV are needed.

HCV is a positive stranded RNA virus of approximately 9,600 bases which encode at least three structural and six non-structural proteins. Based on sequence homology, the structural proteins have been functionally assigned as one single core protein and two envelope proteins: E1 and E2. The E1 protein consists of 192 amino acids and contains 5 to 6N-glycosylation sites, depending on the HCV genotype. The E2 protein consists of 363 to 370 amino acids and contains 9-11N-glycosylation sites, depending on the HCV genotype (for reviews see: Major and Feinstone, 1997; Maertens and Stuyver, 1997). The E1 protein contains various variable domains (Maertens and Stuyver, 1997), while the E2 protein contains three hypervariable domains, of which the major domain is located at the N-terminus of the protein (Maertens and Stuyver, 1997). These envelope proteins have been produced by recombinant techniques in Escherichia coli, insect cells, yeast cells and mammalian cells.

NS2, NS3, NS4A, NS4B, NS5A and NS5B are non-structural (NS) proteins. NS3 is about 70 kDa, and has protease and helicase activity. The sequences in NS3 that are essential for the helicase activity also have RNA binding, Mg⁺⁺ binding, and ATP binding properties. Anti-NS3 antibodies often appear first in sero-conversion series. The immuno-reactivity of the NS3 protein seems to be different in the various commercial assays available today.

To date, vaccination against disease has been proven to be the most cost effective and efficient method for controlling diseases. Efforts to develop an efficacious HCV vaccine, however, have been plagued with difficulties. A conditio sine qua none for vaccines is the induction of an immune response in patients. Consequently, HCV antigenic determinants should be identified, and administered to patients in a proper setting. Antigenic determinants can be divided in at least two forms, i.e. lineair and conformational epitopes. Conformational epitopes result from the folding of a molecule in a three-dimensional space. In general, it is believed that conformational epitopes will realize the most efficacious vaccines, since they represent epitopes which resemble native-like HCV epitopes. However, there are seemingly insurmountable problems with culturing HCV, that result in only minute amounts of virions. In addition, there are vast problems with the expression and purification of recombinant proteins, that result in not properly folded proteins. Therefore, the in vivo structure of most HCV proteins is obscure, and hence no solid study on conformational epitopes has been conducted.

In addition, the lack of suitable in vitro cultivation systems and small animal models has severely impeded the development of new antiviral drugs for hepatitis C infections. The chimpanzee is the only available model today for the study of HCV infection, prophylaxis and therapy, but the system only allows to study previously selected compounds.

It has been suggested that the E1 envelope protein needs the E2 envelope protein to reach a proper folding status (Deleersnyder et al., 1997). In addition, it has been suggested that E1 and E2 form heterodimers which may form the basic unit of the viral envelope (Yi et al., 1997). But, Houghton (1997) reported that repeated immunizations with recombinant gpE1E2 (4×25 μg) of 3 chronically HCV-infected chimpanzees did not induce a significant immune response. The induction of an anti-envelope immune response in patients with hepatitis C would indeed be desirable and beneficial to the patient, since higher levels of such antibodies seem to correlate with good response to interferon therapy, and may therefore help the patient to clear the virus (PCT/EP 95/03031 to Maertens et al.). The antibody levels against E1 in chronic HCV carriers are among the lowest of all HCV antibodies, it may therefore be beneficial to raise those antibody levels, and possibly the cellular response, to induce control or even clearance of the infection by the host. Also, higher levels of cellular immunity against E1 seem to correlate with good response towards interferon therapy (Leroux-Roels et al., 1996). Importantly, the above described studies did not rely on native-like E1 peptides.

The most crucial epitopes in NS3 for detection of HCV positive sera are related to conformational epitopes. Apparently, NS3 epitopes are scattered all over the NS3 protein (see also Leroux-Roels et al. 1996; Rehermann et al., 1996, 1997; Diepolder et al., 1995, 1997). In assays foremost the NS3 protein has been employed instead of peptides.

Advances in molecular biology and genetic engineering have made it possible to produce large amounts of protein products using heterologous expression systems. The use of heterologous hosts, however, can lead to differences in the biological and/or structural properties of the recombinant product. Amongst the biochemical modifications that can occur to proteins during or following the synthesis in the cell and the subsequent purification, the formation and sustainment of disulphide bonds is of importance. Cysteine redox status is intricately linked to the correct folding or assembly of disulphide-bonded proteins. Moreover, very often the biological function of a protein is regulated or at least influenced by the state of oxidation of its sulfhydryl groups. This is the case for some enzymatic activities where the reversibility and timing of oxidation of sulfhydryl groups has been proposed as a physiological control mechanism (see also Thomas et al., 1995; Nakamura et al., 1997; Aslund and Beckwith, 1999).

Several protein factors that catalyze the cysteinyl redox status (thiol versus disulphide bond formation) have been characterised (Mossner et al, 1998; Prinz et al, 1997; Loferer & Hennecke, 1994). Predominantly, these protein factors belong to the “thioredoxin protein superfamily”, of which the members contain 2 redox-active cysteines in the Cys-X-X-Cys consensus sequence (X=any amino acid). This superfamily can be divided in different classes on the basis of the redox potential of the active site, substrate specificity or biological activity. Another classification relies on the consensus sequence of the redox-active centre, namely:

(i) One class, commonly represented by Thioredoxin (TRX), consists of small ubiquitous proteins. The redox-active centre has the consensus sequence Cys-X-Pro-Cys, that is highly conserved in many species, ranging from bacteria to plants and mammals (X=any amino acid). Oxidised TRX_{OX} is regenerated to its reduced form in a complex with TRX-reductase, FAD and NADPH.

(ii) Glutharedoxine (GRX) is a common representative of a second class of the thioredoxine superfamily. The redox-active centre has the consensus sequence Cys-Pro-X-Cys, in which X is preferentially an aromatic amino acid, ie Tyr or Phe. GRX as well TRX act both as reductants with disulfides, but GRX would be a specific glutathion (GSH)-mixed disulfide reductase, e.g. in the reduction of thiolated proteins.

It has been demonstrated that the CXXC motif may also be involved in various intra- and extracellular biochemical and biological functions, eg thiol/disulfide exchange reactions, binding of transition metals, lipid incorporation site, and regulatory activities, such as, for example, control of gene transcription, regulation of signal transduction, including functioning as a cytokine, and the like, and control of the (de)thiolation status of proteins. Importantly, the CXXC motif can function in tertiary as well as quaternary protein structures (see also Thomas et al., 1995; Pinter et al., 1997; Aslund and Beckwith, 1999; Nakamura et al., 1997).

HCV proteins contain CXXC motifs. However, to date there is no suggestion nor indication in the prior art, that the reversible redox status of these CXXC motifs is of importance to HCV. Purification protocol described to date do not account for a reversible —S—S-bridge in the CXXC motif. As a consequence, the conformation of purified HCV proteins as well as their biological activity are impaired.

There have been numerous attempts to study native HCV proteins. The problem encountered was the inability to purify HCV proteins with the correct or native-like conformation. Consequently, conformational epitopes as well as other biochemical and biological functions and activities dependent on the native-like conformation remain enigmatic. In addition, drug targets for liver diseases and viral hepatitis suffer from the same shortcoming, and drug screening programs are bound to fail.

### SUMMARY OF THE INVENTION/AIMS

It thus appears that due to the lack of or inefficient expression and purification systems the correct folding or assembly of proteins is impaired. Such purified proteins are often not biologically active and/or have an incorrect protein structure. As a consequence, native anti-HCV antibodies fail to recognize an important subset of antigenic determinants on these proteins, see for example Houghton (1997).

The present invention overcomes these problems, since it describes and makes for the first time HCV proteins with a native-like conformation, due to a reversible redox status of cysteinyl residues. Thus, new structures of HCV proteins are disclosed. In particular, the present invention allows for the purification of HCV proteins that are biologically active and/or have a native-like conformation. The native-like HCV proteins result in new conformational and oligomerisation-dependent epitopes.

The direct or indirect (mediated) in vitro and in vivo activities of the native-like HCV proteins create the possibility to study biochemical and biological pathways and cascades, eg. metabolic, enzymatic, signal-transduction, immuno-reactivity. The identification of active centres, binding sites and interaction domains (protein-protein, protein-sugar, protein-nucleic acid and protein-small molecule) allow for the development of drugs, that interfere with the cellular and viral processes involved in hepatitis.

the purification and folding method of the present invention, in which a cysteinyl shielding group is removed, followed by refolding and reoxidation of the cysteine residues in the HCV protein, allows to restore the native-like conformation of HCV proteins;

### AIMS

The present invention aims at an HCV protein, or any functionally equivalent part thereof, comprising a Cys-amino acid, which has a reversible redox status. In particular, the present invention pertains to an HCV protein, which comprises at least two Cys-amino acids with a reversible redox status. The latter Cys-amino acids can be spaced by other amino acids.

Preferentially said Cys amino acids are comprised in the amino acid sequence Cys-X₁-X₂-Cys, in which amino acid X₁ denotes any amino acid, and amino acid X₂ denotes any amino acid. More preferentially, amino acid X, denotes either amino acid Val, Leu or lie, and amino acid X₂ denotes amino acid Pro.

Moreover, the present invention aims at providing an HCV protein, or any functionally equivalent part thereof, comprising at least two Cys-amino acids, with a reversible redox status, according to above, obtainable by the following process:

(a) purifying an HCV protein, or any functionally equivalent part thereof, in which the cysteine residues are chemically and/or enzymatically reversibly protected,

(b) removal of the reversibly protection state of the cysteine residues,

(c) obtaining an HCV protein, or any functionally equivalent part thereof, in which the cysteine residues have a reversible redox status.

Moreover, the present invention aims at providing the HCV protein, or any functionally equivalent part thereof, as defined above, for use as a medicament.

Moreover, the present invention aims at the use of the HCV protein, or any functionally equivalent part thereof, as defined above, for the manufacture of an HCV vaccine composition, in particular a therapeutic vaccine or a prophylactic vaccine.

Moreover, the present invention aims at providing the HCV protein, or any functionally equivalent part thereof, as defined above, for raising specific antibodies.

In addition, the present invention aims at providing an immunoassay for detecting HCV antibody by determining formation of an HCV antibody-HCV protein complex.

Finally, the present invention aims at providing a bioassay for identifying compounds that modulate the activity of HCV proteins as defined above, by monitoring changes in oxido-reductase activity.

All the aims of the present invention are considered to have been met by the embodiments as set below.

### FIGURE LEGENDS

FIG. 1: Size exclusion of reversibly protected and irreversibly blocked Vero samples after lysis in the presence of L-ascorbate.

Vero Cells were lysed with Triton X-100 in the presence of 1 mM L-ascorbate. The lysate was loaded on Lentil Lectin and reduced with 7.5 mM DTT at pH 7.2 as described in PCT EP95/03031 to Maertens et al. The reduced E1s was either (1) sulfonated with sodium tetrathionate, (2) irreversibly blocked with N-ethylmaleimide or (3) left untreated but the pH of the solution was decreased to 6.

A SEC profile following the protocol by PCT EP95/03031 to Maertens et al. is included as reference.

The gel filtrations on Superdex G200 10/30 (Pharmacia) were run in PBS, pH 7.2, 3% Empigen, except for condition (3). This gel filtration was run at 10 mM phosphate, 150 mM NaCl, pH 6.0.

SEC Profiles:

A: lysis in presence of ascorbate and sulfonation after reduction with DTT

B: lysis in presence of ascorbate and irreversibly blocking after reduction with DTT

C: lysis in presence of ascorbate and without further treatment, but SEC was run at pH 6.0

D: reference: blocking with NEM/NEM.bio in lysate and after DTT reduction (PCT EP95/03031 to Maertens et al.)

The bars indicate the pools for analysis by silver staining and Western blotting

The histogram gives the sandwich ELISA results: Mab 14H11B2 (IGH 207) was used for coating and the detection was performed with HRP labeled 25C3 (IGH 200).

FIG. 2: Size exclusion chromatography of reversibly protected and irreversibly blocked Vero E1s after lysis in the presence of sulfonation agents

Vero cells were lysed as described in PCT EP95/03031 to Maertens et al., but sodium tetrathionate was added instead of NEM/NEM.bio.

The purification on lentil and reduction were performed as described in PCT EP95/03031 to Maertens et al. The reduced material was either (1) sulfonated by sodium tetrathionate either (2) treated with IAA (=irreversibly blocked).

The material obtained by the method as described in PCT EP95/03031 to Maertens et al. is included as reference.

The 3 different E1s samples were separated on a Superdex G200 10/30 column, which had been equilibrated with PBS, 3% Empigen, pH 7.2.

A: Sulfonation of the Vero cell lysate and sulfonation after reduction with DTT

B: Sulfonation of the Vero cell lysate and irreversible blocking with Iodo-acetamide

C: Vero E1s obtained after irreversible blocking with NEM/NEM.bio as described in PCT EP95/03031 to Maertens et al.

D: overlay of the SEC profiles

The results of the sandwich ELISA are presented in the histograms.

The E1s-fractions were pooled as indicated with the bars and analysed by silver staining and Western blot.

FIG. 3: Fraction analysis of the SEC in 3% Empigen by SDS-PAGE and Western blotting.

(A): SEC Fractions obtained after the different conditions of reversible protection and irreversible blocking were analysed by SDS-PAGE and silver staining.

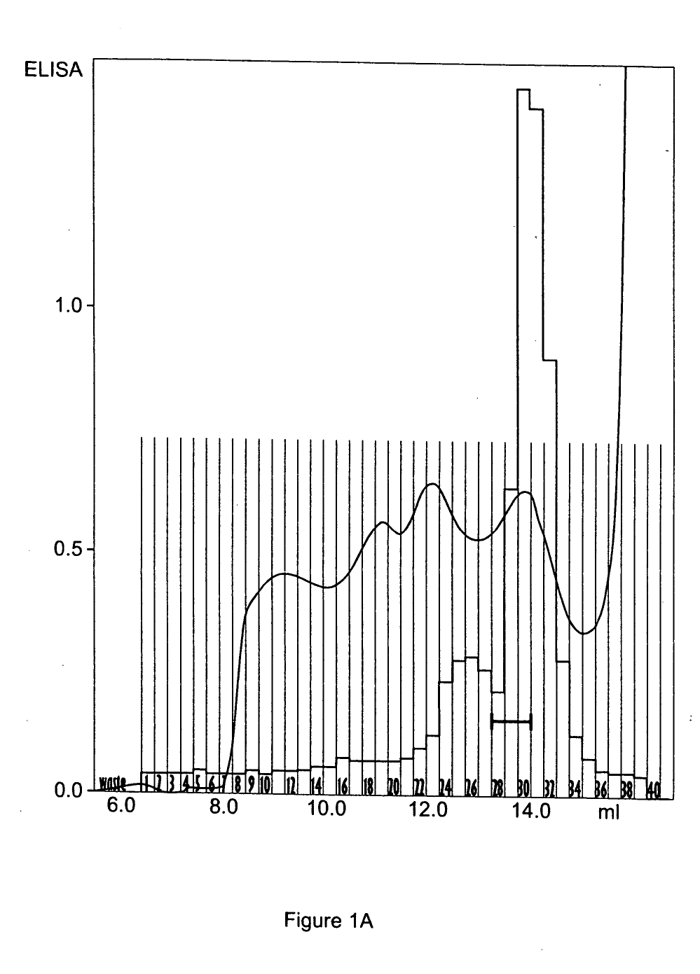

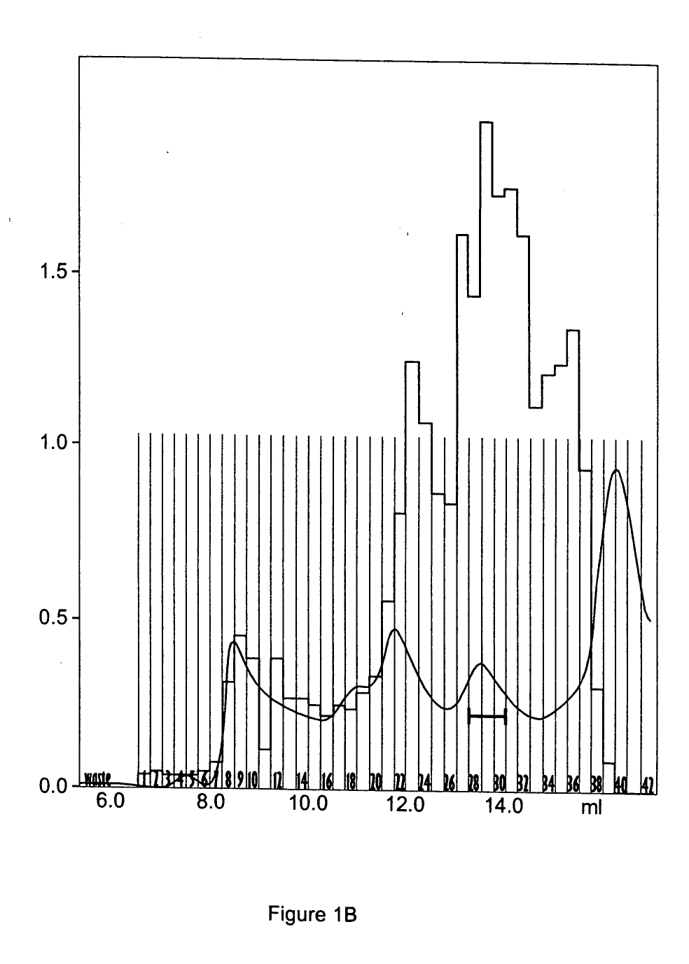

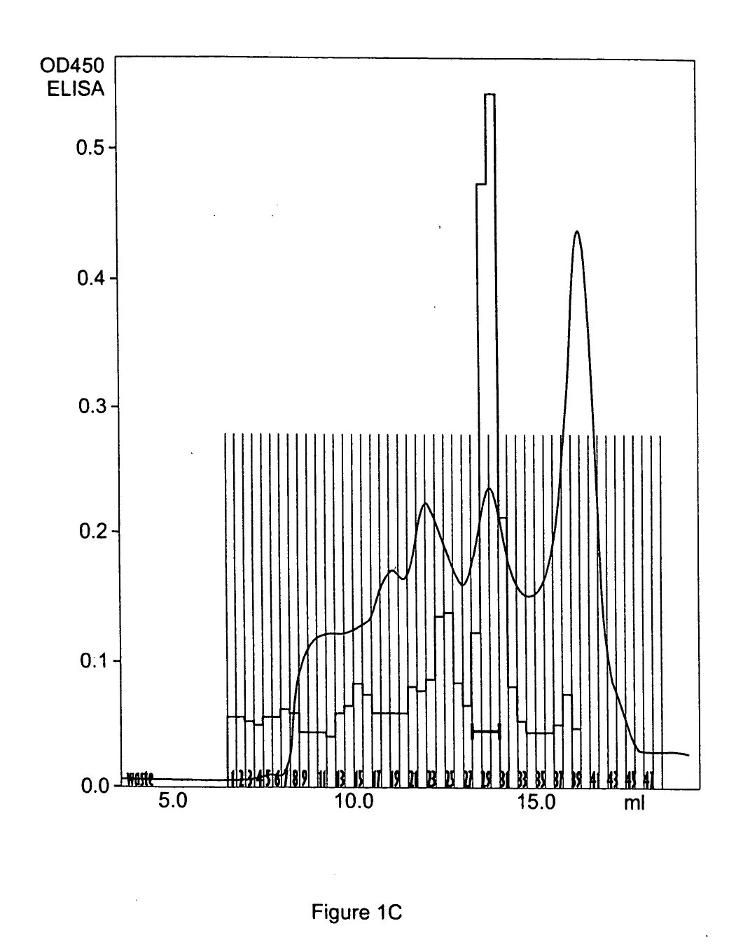

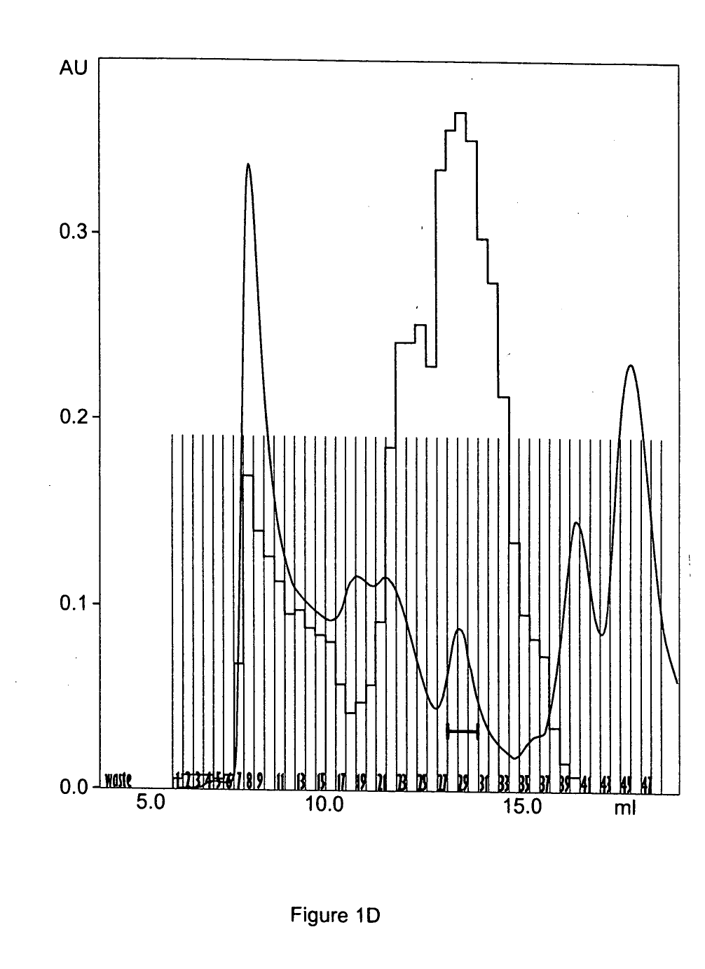

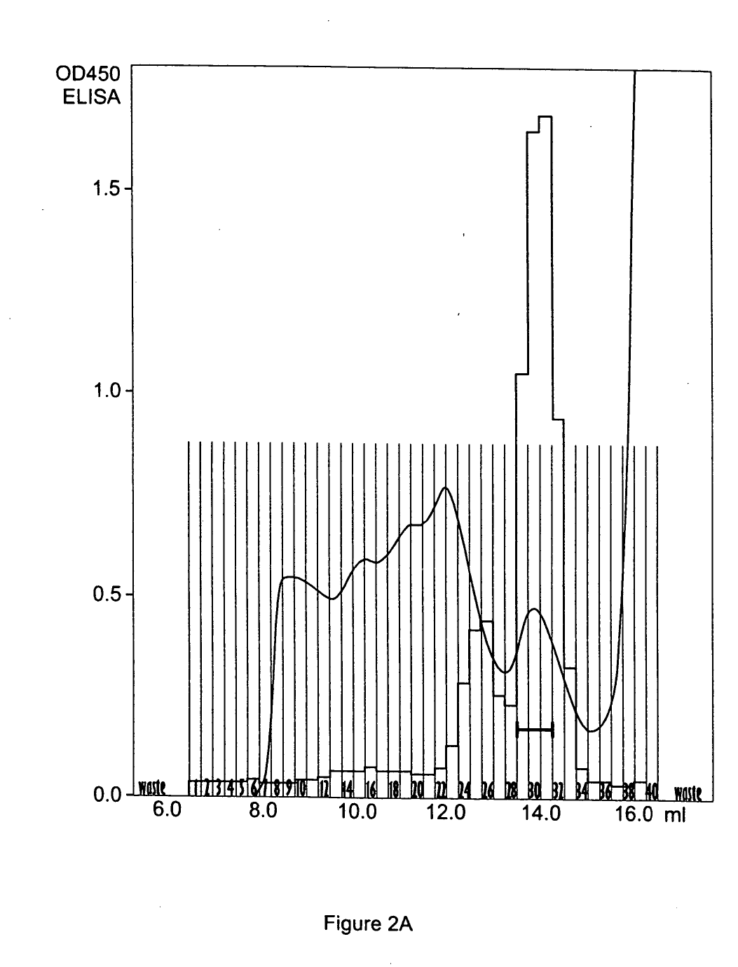

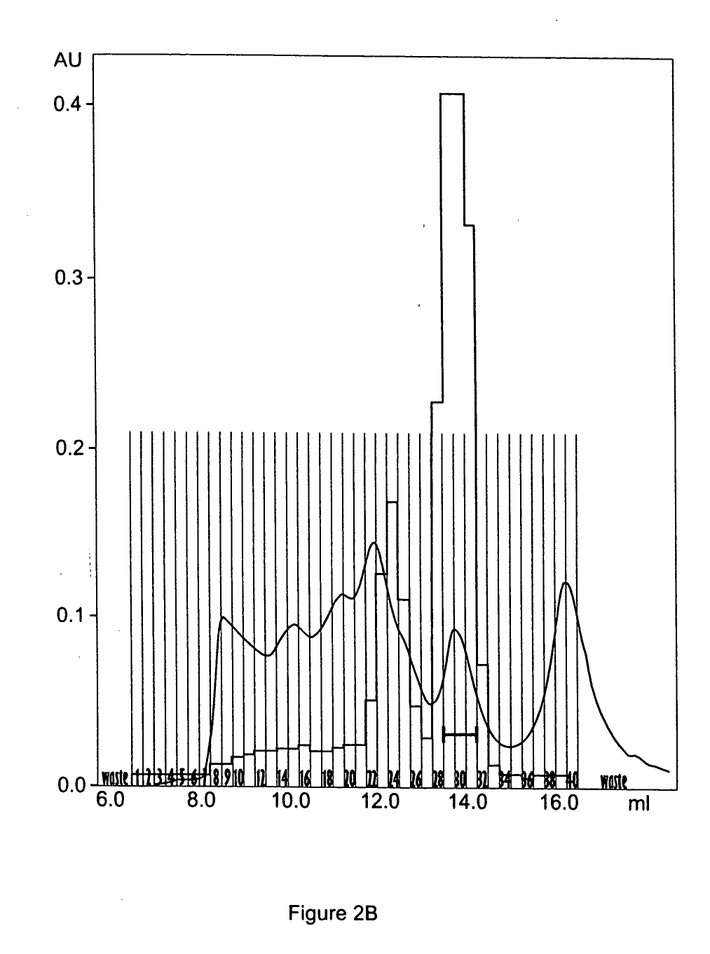

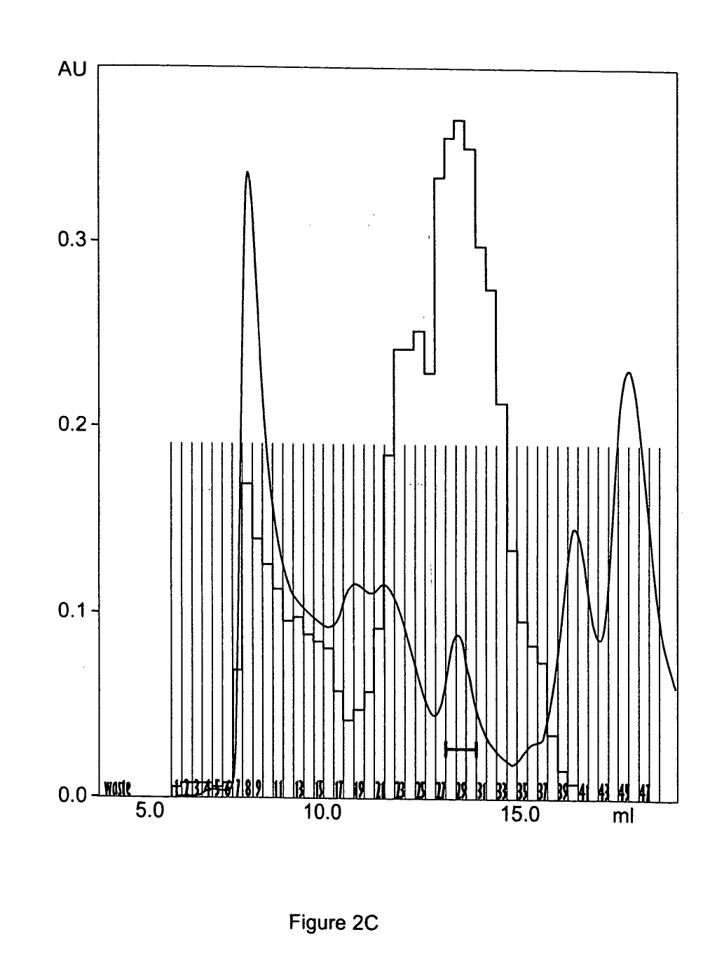

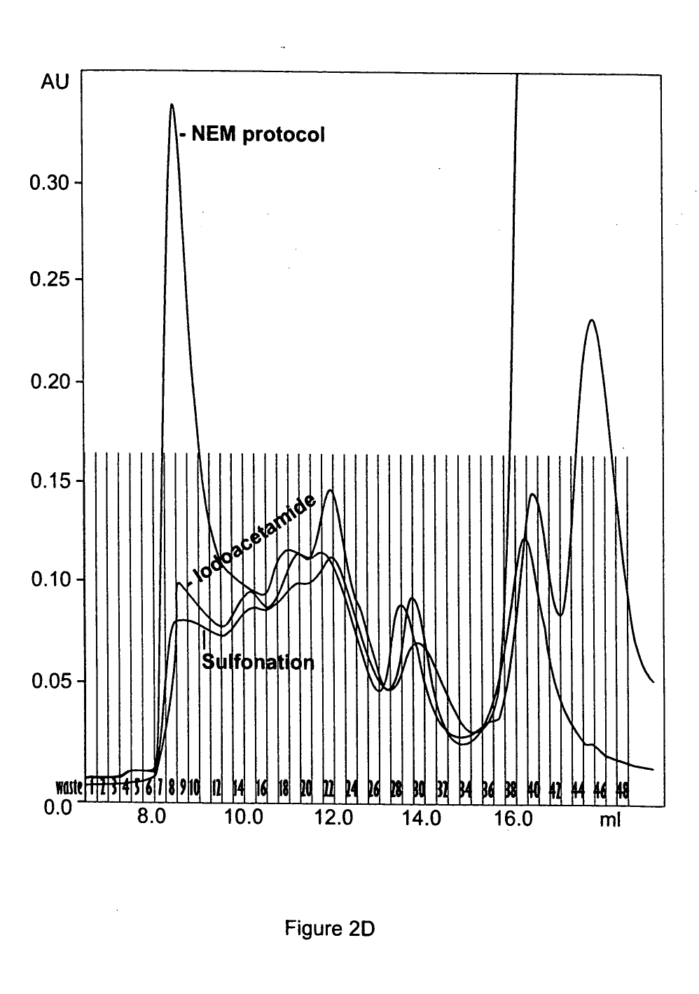

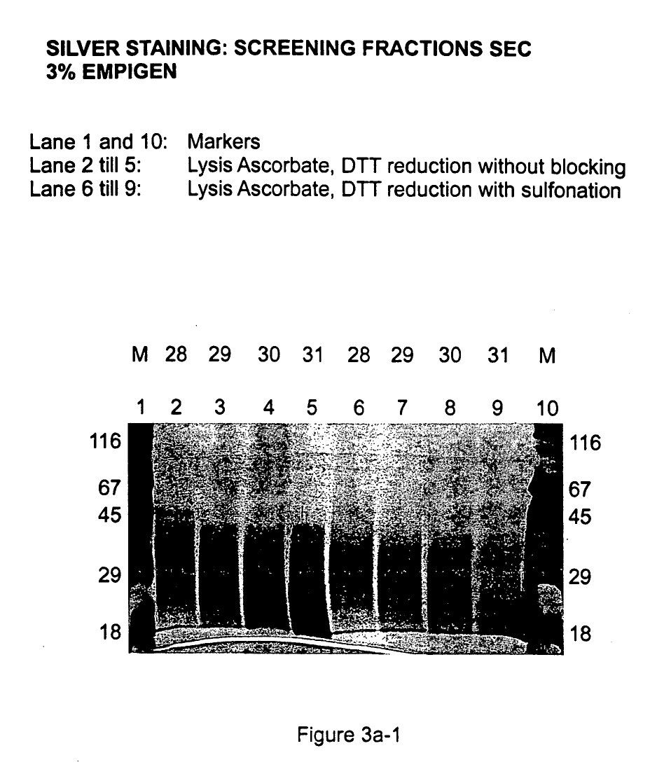

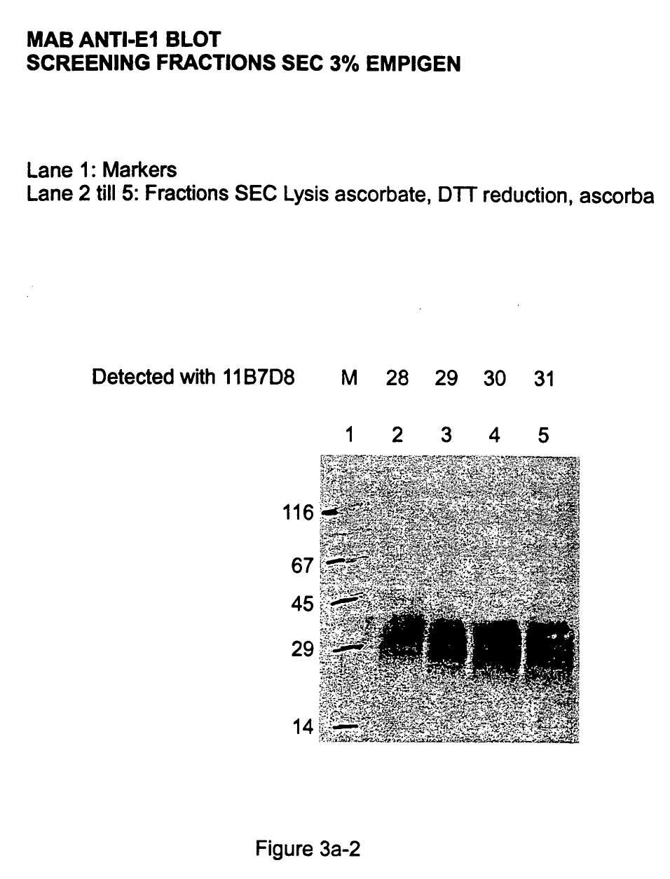

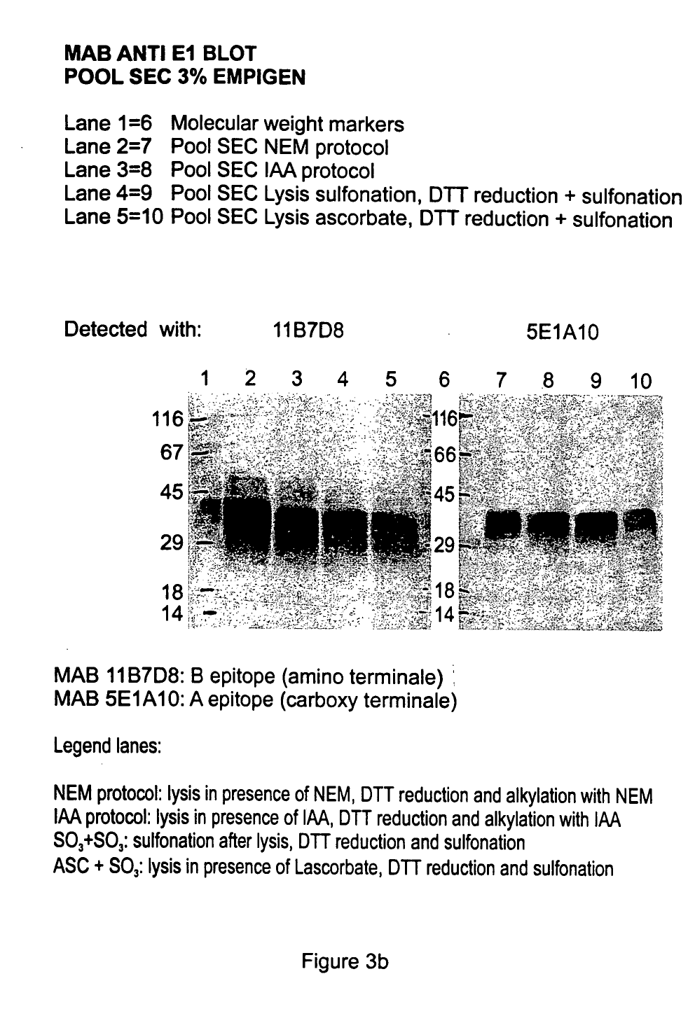

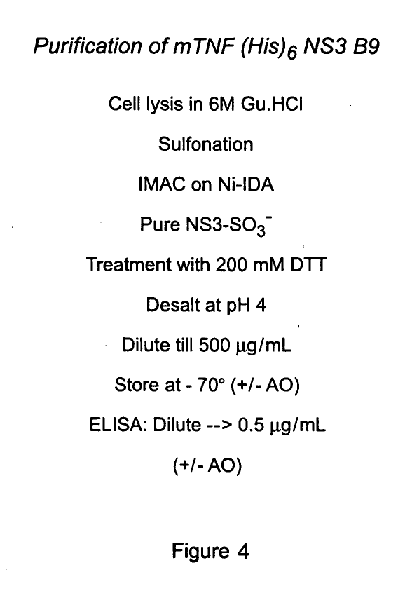

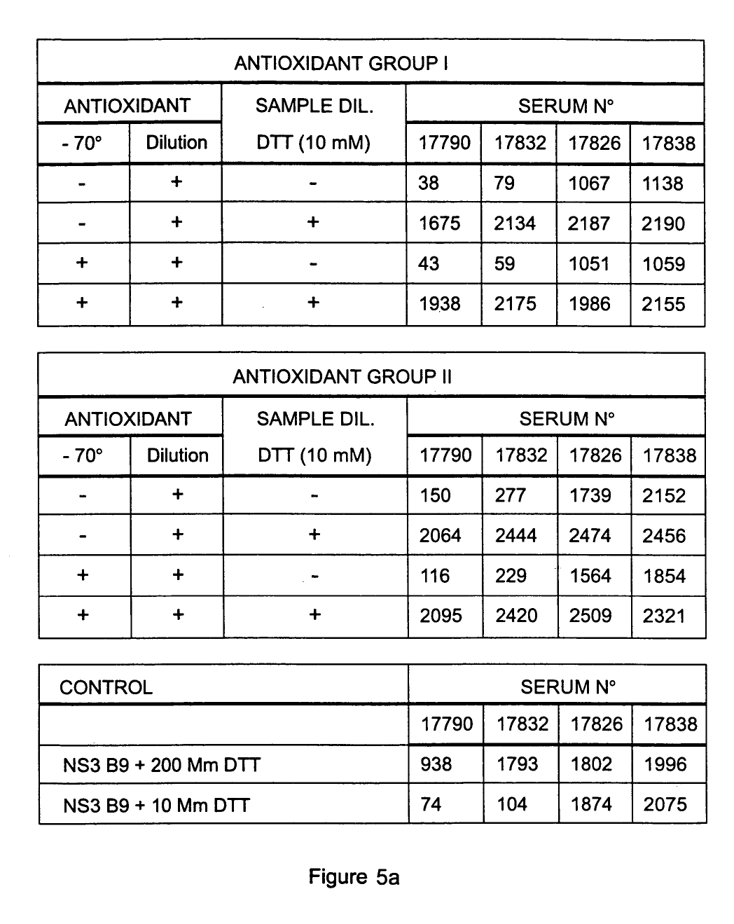

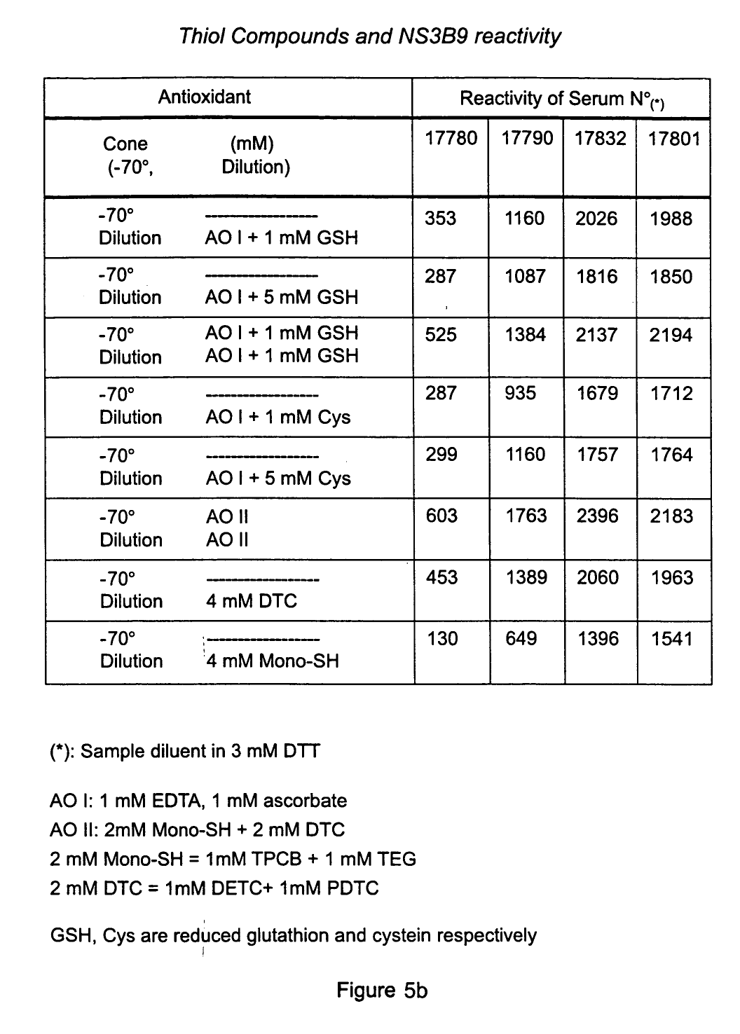

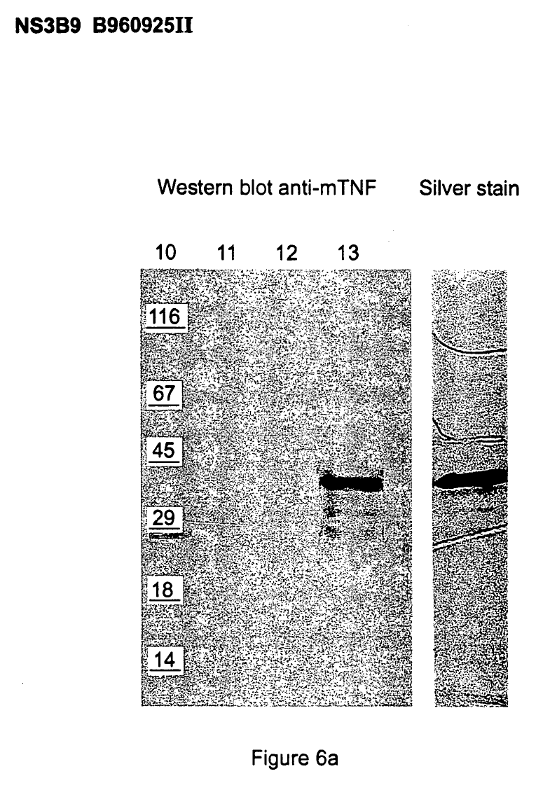

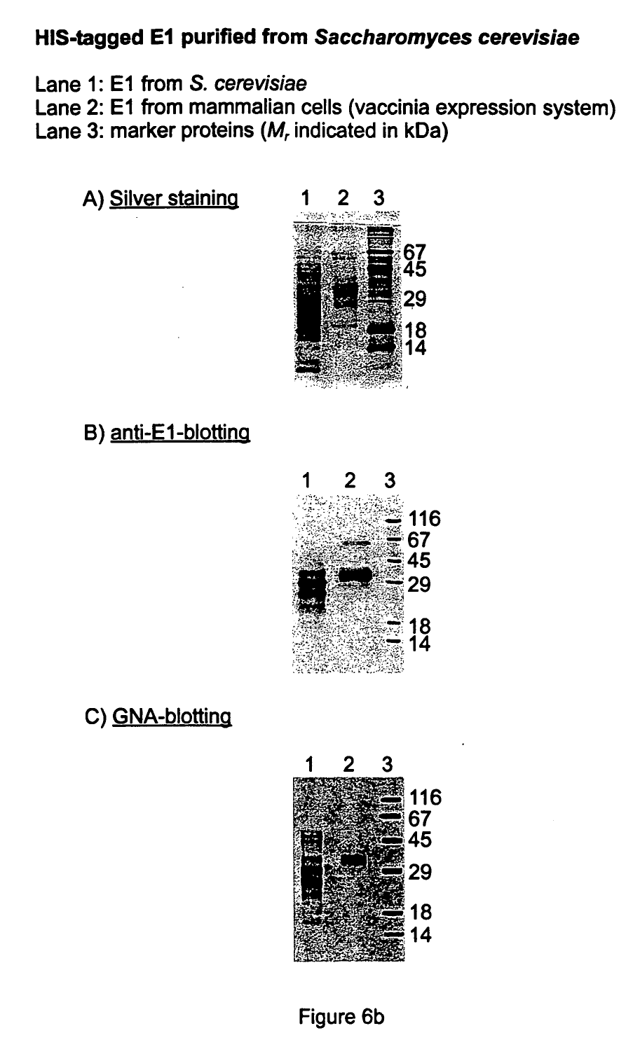

SDS-PAGE analysis of Fractions obtained after lysis in ascorbate and gel filtration at pH 6 (see FIG. 1.c) or lysis in ascorbate and sulfonation (FIG. 1.a) are given as examples in FIG. 3A.1.

FIG. 3A.2 shows the fraction screening by Western blot with 11B7D8 for the conditions described as in FIG. 1.C (lysis in ascorbate and SEC at pH 6 after DTT reduction).

(B) Western blots of the SEC-pools were performed with anti-E1s MAbs 5E1A10, which recognizes the amino- and carboxy-terminal epitope respectively.

The pools were made as indicated in FIG. 1 and FIG. 2.

Lane 1 and 6: Molecular weight markers

Lane 2 and 7: reference material as prepared by PCT EP95/03031 to Maertens et al.

Lane 3 and 8: reference material prepared with irreversibly blocked cysteines (Treatment with Iodo-acetamide)

Lane 4 and 9: material obtained after sulfonation of lysate and sulfonation after reduction

Lane 5 and 10: material obtained after lysis in the presence of ascorbate and sulfonation after reduction.

FIG. 4 E. coli expressed (his)₆—tagged NS3 fusion protein

Purification on metal affinity after reversibly protection as well as sample preparation for ELISA is schematized.

+/−AO: in the presence or absence of reversible protecting agent (AO)

FIG. 5 ELISA reactivity of the mTNF(His)₆ NS3 fusion proteins after different coating conditions.

FIG. 5A: 90% pure mTNF(His)₆NS3B9 fusion protein was desalted to 25 mM citrate, 1 mM EDTA, pH 4 after reduction with 200 mM DTT. The fusion protein was diluted till 500 μg/mL in desalting buffer and stored at −70° C. in the presence or absence of thiol protective agents (antioxidant group 1, group 2).

The samples were diluted to 0.5 μg/mL in ELISA coating buffer (50 mM bicarbonate buffer, pH 9.6) with or without thiol protecting agents (anti-oxidant). The wells were blocked with PBS in presence or absence of protecting agent.

Serum sample incubation was performed in presence or absence of 10 mM DTT and the ELISA was developed with HRP conjugated rabbit anti human antibodies (Dako, Denmark) after washing. The reaction was stopped by addition of 2N H₂SO₄.

Sera (17790, 17826, 17832, 17838) were tested. Sera 17790 and 17832 are considered as difficult sera, because they are only detected as HCV positive sera after treatment with 200 mM DTT (positive control). The 10 mM DTT treatment is included as negative control for these sera. Sera 17826 and 17838 are sera, that react with the NS3B9 protein after 10 mM DTT treatment (and are considered as easily detectable HCV sera).

Antioxidant group 1:1 mM EDTA, 1 mM L-ascorbic acid, 1 mM reduced glutathion. 1 mM tocopherol was supplementary added to these thiol protecting agents during the ELISA process, if the blocking was performed in the presence of protective agent.

Antioxidant group 2: 1 mM thiodiethyleneglycol (TEG), 1 mM thiophenecarboxylic acid (TPCB), 1 mM pyrrolidone dithiocarbamate (PDTC), 1 mM diethyl dithiocarbamate (DETC).

FIG. 5B: Thiol Compounds and NS3 B9 reactivity

The ELISA was performed as described in FIG. 5A, except that the effect (type and concentration) of mono- and dithio compounds as reversible protection group was investigated in more detail.

The sample diluent was incubated in this ELISA always in the presence of 3 mM DTT.

Antioxidant 1=1 mM EDTA, 1 mM L-ascorbate

Antioxidant 2=1 mM thiophenecarboxylic (TPBC) acid, 1 mM thioethyleneglycol (TEG), 1 mM Diethyl dithiocarbamate (DETC), 1 mM pyrrolidone dithiocarbamate (PDTC).

4 mM DTC=2 mM DETC, 2 mM PDTC

4 mM Mono-SH=2 mM TPBC, 2 mM TEG.

GSH and Cys are reduced glutathion and cysteine respectively.

FIG. 6 SDS-PAGE analysis of purified reversible protected (his)₆-tagged HCV proteins after metal affinity chromatography

6A: E. coli expressed mTNF(His)₆NS3B9 (batch NS3 B9 B96092511).

Western blot with anti mTNF and silver stained SDS-PAGE under non reducing conditions. (1 μg protein/lane).

6B: Saccharomyces cerevesiae (Yeast) expressed (his)₆-tagged E1s

The proteins were visualised by (a) silver staining; (b) Western blotting anti E1s or (c) GNA blotting.

Vaccinia expressed E1s, purified as described by Maertens et al (PCT EP95/03031) was included as reference.

### DETAILED DESCRIPTION OF THE INVENTION

The invention described herein draws on previously published work and pending patent applications. By way of example, such work consists of scientific papers, patents or pending patent applications. All these publications and applications, cited previously or below are hereby incorporated by reference.

The present invention relates to HCV proteins with specific conformations. For the first time HCV proteins with a native-like conformation are generated, in particular HCV E1 protein. Specific cysteine bonds involved in the conformation of these HCV proteins were found to be important. As a way of example, a new and inventive purification protocol is disclosed that enables to purify HCV proteins with a native-like conformation. These new HCV proteins are able to not only present conformational epitopes but also display biological activity. These new HCV proteins can be used for various studies, such as, for example, studies on drug screening, biological activities, signal-transduction pathways, intra- and extracellular processing, interactions and binding between HCV and/or non-HCV molecules, oligomerisation, conformational epitopes, antibody screening, metabolism and zenzymatic activity, immuno-reactivity. Apparently, these studies can be placed in a context for an eventually diagnostic and/or therapeutic application.

The present invention is based on the finding that HCV proteins have specific, native-like conformations and biological activity, due to reversible redox status of cysteinyl residues.

The present invention pertains therefore to an HCV protein, or any functionally equivalent part thereof, comprising a Cys-amino acid, which has a reversible redox status. In particular, the present invention pertains to an HCV protein, which comprises at least two Cys-amino acids with a reversible redox status. The latter Cys-amino acids can be spaced by other amino acids. Preferentially said Cys amino acids are comprised in the amino acid sequence Cys-X₁-X₂-Cys, in which amino acid X₁ denotes any amino acid, and amino acid X₂ denotes any amino acid. More preferentially, amino acid XI denotes either amino acid Val, Leu or lie, and amino acid X₂ denotes amino acid Pro.

HCV

In this regard, the present invention relates to HCV, and other members of the genus Flaviviridae, such as, for example, Hepatitis G virus, Dengue virus, Yellow Fever Virus. Thus, the term “HCV” contemplates all members of the genus Flaviviridae.

Protein

The term “protein” as used herein, refers to an HCV protein, or any functionally equivalent part thereof, containing in its amino acid sequence at least one cysteine, the redox status of which is variable (see below). Also, protein “domains” containing at least one cysteine in its amino acid sequence are contemplated in the term “protein”. The term “functionally equivalent part thereof” as used herein refers to a part or fragment of said HCV protein that contains in its amino acid sequence at least one cysteine, the redox status of which is variable. In particular, the terms “protein” and “functionally equivalent part thereof” refers to HCV proteins and fragments thereof comprising a redox active center, such as, for example, HCV E1 protein. More particularly, the present invention relates to HCV E1s, and HCV E1p. In this regard, the term “redox active center” as used herein connotates a protein motif with the consensus sequence CXXC.

The term “a peptide” refers to a polymer of amino acids (aa's) derived from the well-known HCV proteins (Linnen et al., 1996; Maertens and Stuyver, 1997). The term “HCV E1” is a well-known protein by a person skilled in the art (Wengler, 1991). HCV E1, together with HCV E2, which was previously called non-structural protein 1 (NS1) or E2/NS1, constitute the envelope region of HCV.

| | |
|---|---|
| HCV E1s (192-326) | |
| YEVRNVSGMY HVTNDCSNSS IVYEAADMIM HTPGCVPCVR ENNSSRCWVA | (SEQ ID NO 1) |
| LTPTLAARNA SVPTTTIRRH VDLLVGAAAF CSAMYVGDLC GSVFLVSQLF | |
| TISPRRHETV QDCNCSIYPG HITGHRMAWD MMMNW | |
| HCV E1p (192-237) | |
| YEVRNVSGMY HVTNDCSNSS IVYEAADMIM HTPGCVPCVR ENNSSR | (SEQ ID NO 2) |

The term “peptide” refers to a polymer of amino acids and does not refer to a specific length of the product. The terms “peptide”, “polypeptide”, “polyprotein” and “protein” are thus included within the definition of “peptide”, and are used interchangeably herein. The term “peptide” does not refer to or exclude post-expression modifications of the peptide, for example, glycosylations, acetylations, phosphorylations, and the like. Included within the definition of peptide are, for example, polypeptides containing one or more analogues of an amino acid (including, for example, unnatural amino acids, PNA (Nielsen et al., 1991, 1993), etc.), peptides with substituted linkages, as well as other modifications known within the art, both naturally occurring and non-naturally occurring. Hence, peptides may be linear, circular or constrained (cyclised or stabilised by ‘S—S’ bridges, other than according to the present invention), consisting of D- or L-amino acids; peptides may be multimeric, branched, presented on phages or immobilised covalently or non-covalently on polymers from different nature, such as, for example, organic, lipid, carbohydrate, protein, nucleic acid polymers; or peptides may be present in a scaffold. It is thus to be understood that peptidomimitics or mimotopes are inherent in the terms “polypeptide”, “peptide” and “protein”.

Immobilisation on polymers can be realised by residues of the HCV peptide self or by HCV peptide fused or coupled to other molecules such as, for example via a his-tag (Dietrich et al., 1996) or lipid chelators (Dietrich et al., 1995)

The term “mimotopes” refers to polypeptides which mimic the polypeptides as defined herein immunologically. Since sequence variability has been observed for HCV, it may be desirable to vary one or more amino acids as to better mimic the epitopes of different strains. It should be understood that such mimiotopes need not be identical to any particular HCV sequence as long as the subject compounds are capable of providing for immunological competition with at least one strain of HCV.

The term “peptidomimitics” refers to molecules that do not need to be composed solely of amino acids, but mimic the polypeptides as defined herein immunologically.

The present invention specifically refers to peptides prepared by classical chemical synthesis. The synthesis can be carried out in homogeneous solution or on solid phase. For instance, the synthesis technique in homogeneous solution which can be used is the one described by Houbenweyl (1974). The peptides of the present invention can also be prepared by solid phase according to the methodes described by Atherton and Shepard (1989). In addition, HCV peptides, peptidometics and mimotopes synthesized by dendrimer (Zhang & Tam, 1997), polyketide (Carreras & Santi, 1998) or intein technology (Southworth et al, 1999) are also included in the present invention.

The peptides according to the present invention can also be prepared by means of recombinant DNA techniques, such as described in Sambrook et al. (1989), in prokaryotes or lower or higher eukaryotes. The term ‘lower eukaryote’ refers to host cells such as yeast, fungi and the like. Lower eukaryotes are generally (but not necessarily) unicellular. The term ‘prokaryotes’ refers to hosts such as E. coli, Lactobacillus, Lactococcus, Salmonella, Streptococcus, Bacillus subtilis or Streptomyces. Also these hosts are contemplated within the present invention. Preferred lower eukaryotes are yeasts, particularly species within Schizosaccharomyces, Saccharomyces, Kluiveromyces, Pichia (e.g. Pichia pastoris), Hansenula (e.g. Hansenula polymorpha), Schwaniomyces, Schizosaccharomyces, Yarowia, Zygosaccharomyces and the like. Saccharomyces cerevisiae, S. carlsbergensis and K. lactis are the most commonly used yeast hosts, and are convenient fungal hosts. The term ‘higher eukaryote’ refers to host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals.

The proteins according to the present invention can also be isolated from mammalian hosts, in particular mice or primates, e.g. humans as well as non-humans.

It is well known in the art that amino acids can be denoted by their full name, three-letter abbreviation, and one-letter symbol (see eg Stryer, 1981).

Furthermore, the present invention pertains to an HCV protein or part thereof as defined above, which specifically binds intra- or intercellular host molecules (host-derived molecules), such as, for example,

(i) receptor proteins, eg. annexin V, apolipoprotein B, tubulin, 24 kDa plasma membrane protein (Abrigani WO 97/09349), mannose receptor, asialoglycoprotein receptor;

(ii) molecules (protein or non-protein compounds) involved in redox regulation, eg. Gluthathion, TRX and GRX;

(iii) chaperone proteins, eg calnexin;

(iv) various glycoseaminoglycans (peptide and/or sugar core);

(v) nucleic acids or lipids.

Furthermore, the present invention pertains to an HCV protein or part thereof as defined above, which specifically binds another HCV protein or HCV nucleic acid (HCV-derived molecules), or parts thereof, resulting in homo- and/or hetero-oligomeric complexes.

The complexes resulting from HCV proteins, or parts thereof, as defined above bound to other HCV-derived molecules or host-derived molecules are colloquially denoted “HCV-derived complex”. Thus, an “HCV-derived complex” consists of at least an HCV protein as defined above connected to another molecule, ie (HCV-protein)-X, in which X is a host-derived molecule or an HCV-derived molecule.

Pure

The term “purified” as applied herein refers to a composition wherein the desired components, such as, for example, HCV envelope proteins, comprise at least 35% of the total components in the composition. The desired components preferably comprises at least about 40%, more preferably at least about 50%, still more preferably at least about 60%, still more preferably at least about 70%, even more preferably at least about 80%, even more preferably at least about 90%, even more preferably at least about 95%, and most preferably at least about 98% of the total component fraction of the composition. The composition may contain other compounds, such as, for example, carbohydrates, salts, lipids, solvents, and the like, without affecting the determination of the percentage purity as used herein. An “isolated” HCV protein intends an HCV protein composition that is at least 35% pure. In this regard it should be clear that the term “a purified HCV protein” as used herein, refers to isolated HCV proteins in essentially pure form. The term “essentially purified HCV proteins” as used herein refers to HCV proteins such that they can be used for in vitro diagnostic methods and therapeutics. These HCV proteins are substantially free from cellular proteins, vector-derived proteins or other HCV viral components. Usually, these proteins are purified to homogeneity, at least 80% pure, preferably 85%, more preferably 90%, more preferably 95%, more preferably 97%, more preferably 98%, more preferably 99%, even more preferably 99.5%, and most preferably the contaminating proteins should be undetectable by conventional methods such as SDS-PAGE and silver staining.

Antibodies

The present invention relates also to an HCV-antibody that can recognise an HCV-peptide as described above.

Furthermore, the present invention relates to an HCV protein or a functionally equivalent part thereof as defined supra, for raising anti-HCV antibodies, that specifically recognise said HCV protein or a functionally equivalent part thereof.

The term an “HCV-antibody” refers to any polyclonal or monoclonal antibody binding to an HCV-protein of the present invention or an HCV-derived complex.

Moreover, the term “HCV-antibodies” also connotates specific HCV-antibodies that are raised against epitopes which result from the conformation in HCV proteins due to the presence of S—S-bridges in these HCV proteins. Notably, said S—S-bridges can be an intrinsic be part of the epitope. But the oxido-reduction status of the cysteines (reduced or oxidised; in a thiolated or S-conjugated form) may change or stabilise the protein conformation (in the vicinity or not of these cysteine residues) which result in new epitopes, that may or may not contain these cysteine residues. These new epitopes are also part of the invention. In addition, the term an “HCV antibody” refers also to any polyclonal or monoclonal antibody binding to mimitopes, as defined above.

In addition, the term “HCV-antibody” thus also pertains to antibodies that bind antigenic determinants resulting from the specific conformation of HCV-derived complexes, ie antibodies that bind antigenic determinants which are not present on either the HCV-peptide of the present invention or the molecule said HCV-peptide is bound to, such as, for example, epitopes that find their origin from the interaction between the HCV peptide of the present invention and non-protein compounds like glycosaminoglycans (GAGs), heparine, nucleic acids, lipids, cofactors like metal-ions, and the like. Moreover, antigenic determinants may be formed by conformational changes, such as for example introduced by protein processing, cleaving or pH changes.

The term “epitope” refers to that portion of the antigen-antibody complex that is specifically bound by an antibody-combining site. Epitopes may be determined by any of the techniques known in the art, or may be predicted by a variety of computer prediction models known in the art.

The expressions “recognising”, “binding”, or “formation of an antibody-protein complex” as used in the present invention is to be interpreted that binding, i.e. interaction, between the antigen and the antibody occurs under all conditions that respect the immunological properties of the antibody and the antigen.

Moreover, there are various other procedures known to produce HCV peptides, that differ from the procedure of the present invention. These other procedures might result in HCV peptides capable of presenting epitopes. It is conceivable that the HCV peptides, obtained by these various and different procedures, are capable of presenting epitopes similar to the epitopes of the present invention. Thus, similar epitopes are epitopes resulting from different production or purifying procedures than from the present invention, but recognizable by one and the same antibody. However, the proteins of the instant invention present epitopes extremely efficient. Consequently, the epitopes on the proteins are more immunogenic. Therefore, the present invention also pertains to epitopes on proteins, said epitopes are at least 10 times, preferentially at least 20 times, preferentially at least 50, preferentially at least 100 times, preferentially at least 500 times, and most preferentially at least 1000 times more immunogenic than epitopes on HCV-peptides, which are not produced according to the present invention, and which do not have cysteinyl residues with a reversible redox status. It will be appreciated by the skilled in the art that said immunogenecity can, for example, be detected and therefore compared by immunising mammals by means of administering comparable quantities of peptides, produced by either method.

More particularly, the term “HCV-antibody” refers to an antibody binding to the natural, recombinant or synthetic HCV proteins, in particular binding to the natural, recombinant or synthetic E1, E1s, E1p and/or NS3 proteins derived from HCV, or any functionally equivalent variant or part thereof (anti-HCV-E1-, anti-HCV-E1s-, anti-HCV-E1p- or HCV-NS3-antibody, respectively). HCV-antibody may be present in a sample of body fluid, and may be an HCV-E1-antibody, HCV-E1s-antibody, HCV-E1p-antibody or HCV-NS3-antibody.

The term “monoclonal antibody” used herein refers to an antibody composition having a homogeneous antibody population. The term is not limiting regarding the species or source of the antibody, nor is it intended to be limited by the manner in which it is made. Hence, the term “antibody” contemplates also antibodies derived from camels (Arabian and Bactrian), or the genus lama.

Thus, the term “antibody” also refers to antibodies derived from phage display technology or drug screening programs.

In addition, the term “antibody” also refers to humanized antibodies in which at least a portion of the framework regions of an immunoglobulin are derived from human immunoglobulin sequences and single chain antibodies as described in U.S. Pat. No. 4,946,778 and to fragments of antibodies such as Fab, F′(ab)₂, Fv, and other fragments which retain the antigen binding function and specificity of the parent antibody. The term “antibody” also refers to diabodies, triabodies or multimeric (mono-, bi-, tetra- or polyvalent/mono-, bi- or polyspecific) antibodies, as well as enzybodies, ie artificial antibodies with enzyme activity. Combinations of antibodies with any other molecule that increases affinity or specificity, are also contemplated within the term “antibody”. Antibodies also include modified forms (e.g. mPEGylated or polysialylated form (Fernandes & Gregoriadis, 1997) as well as covalently or non-covalently polymer bound forms.

In addition, the term “antibody” also pertains to antibody-mimicking compounds of any nature, such as, for example, derived from lipids, carbohydrates, nucleic acids or analogues e.g. PNA, aptamers (see Jayasena, 1999).

HCV antibodies may be induced by vaccination or may be passively transferred by injection after the antibodies have been purified from pools of HCV-infected blood or from blood obtained from HCV vaccinees.

The present invention relates also to a kit comprising HCV-antibodies for detecting the HCV peptides as defined herein.

Purification Procedure

The invention further pertains to a purification procedure as described herein, resulting in HCV proteins of which at least one cysteinyl residue has a reversible redox status, as well as the HCV proteins obtainable by said purification procedure. During purification at least the cysteine residues are reversibly protected by chemical and/or enzymatic means (see also Examples section).

In this regard, the term “reversible redox status” as used herein refers to sulfur of cysteines which have the ability to change from the reduced status to the oxidized status and vice versa. This change in redox status involves electron transfer. The term “oxido-reductase activity” as used herein refers to the redox potential of the redox active center, and thus to its ability to transfer electrons from and to substrate molecules. This ability is dependent of the redox potential of the substrate molecules and the chemical environment.

Native HCV proteins have a specific conformation and may display biological activity. The purification procedure of the present invention results in purified HCV proteins with a biological activity and/or conformation which is identical to or almost identical (native-like) to the native biological activity and/or conformation of HCV proteins. The purification procedure of the present invention is characterised by the following:

-A— The first phase in the purification procedure of the present invention is intended to reversibly protect the reactivity of the cysteine residues.

In essence, the first phase consists of the procedure as described extensively in PCT EP95/03031 to Maertens et al., but for one fundamental difference, in particular the cysteine residues are reversibly protected.

Reversible protection of the cysteine residues can be achieved by one of the following conditions (i) a modification group, or by (ii) stabilisation of the thiols and/or disulfide bridges. In effect, this protection stabilises the HCV protein, i.e. thiols and/or disulfide bridges have no tendency to react.

Hence, the first phase results eventually in a pure product with reversibly protected cysteines;

-B— The second phase in the purification procedure of the present invention is intended to restore the reactivity of the cysteine residues.

The condition in which the cysteine residues are reversibly protected is removed, after the first phase of the purification procedure.

This removal enables the restoration of the reversible redox status of the cysteine residues.

Thus finally, an HCV peptide, or any functionally equivalent part thereof, is obtained in which the cysteine residues have a reversible redox state.

The reversible redox status allows for reactive HCV proteins with biological activity and/or with a native-like conformation.

Therefore, the present invention pertains to an HCV protein, or any functionally equivalent part thereof, comprising at least two Cys-amino acids, which have a reversible redox status, as defined above, obtainable by the following process:

(a) purifying an HCV protein, or any functionally equivalent part thereof, in which the cysteine residues are reversibly protected by chemical and/or enzymatic means,

(b) removal of the reversibly protection state of the cysteine residues,

(c) obtaining an HCV protein, or any functionally equivalent part thereof, in which the cysteine residues have a reversible redox status.

Thus, the present invention pertains also to the latter process.

Optionally, cofactors and antioxidantia are added to aid in protein stabilisation.

It is to be understood that the purpose of reversibly protection is to stabilise the HCV protein. Especially, after reversibly protection the sulfur-containing functional group (eg thiols and disulfides) is retained in a non-reactive condition. The sulfur-containing functional group is thus unable to react with other compounds, e.g. no tendency of forming or exchanging disulfide bonds, such as, for example

R₁—SH+R₂—SH-x->R₁—S—S—R₂;

R₁—S—S—R₂+R₃—SH-x->R₁—S—S—R₃+R₂—SH;

R₁—S—S—R₂+R₃—S—S—R₄-x->R₁—S—S—R₃+R₂—S—S—R₄.

The described reactions between thiols and/or disulphide residues are not limited to intermolecular processes, but may also occur intramolecularly.

The term “reversibly protecting” as used herein contemplates covalently binding of modification agents to the cysteine residue, as well as manipulating the environment of the HCV protein such, that the redox state of the thiol-groups remains unaffected throughout subsequent steps of the purification procedure (shielding).

Reversible protection of the cysteine residues can be carried out chemically or enzymatically.

The term “reversible protection by enzymatical means” as used herein contemplates reversible protection mediated by enzymes, such as for example acyl-transferases, e.g. acyl-transferases that are involved in catalysing thio-esterification, such as palmitoyl acyltransferase (see below and Das et al., 1997).

The term “reversible protection by chemical means” as used herein contemplates reversible protection:

(1) by modification agents that reversibly modify cysteinyls such as for example by sulphonation and thio-esterification;

Sulphonation is a reaction where thiol or cysteines involved in disulfide bridges are modified to S-sulfonate: RSH→RS—SO₃— (Andre Darbre) or RS—SR→2 RS—SO₃— (sulfitolysis; Kumar et al, 1986). Reagents for sulfonation are e.g. Na₂SO₃, or sodium tetrathionate. The latter reagents for sulfonation are used in a concentration of 10-200 mM, and more preferentially in a concentration of 50-200 mM. Optionally sulfonation can be performed in the presence of a catalysator such as, for example Cu²⁺ (100 μM-1 mM) or cysteine (1-10 mM).

The reaction can be performed under protein denaturing as well as native conditions (Kumar et al., 1985; Kumar et al., 1986).

Thioester bond formation, or thio-esterification is characterised by:

RSH+R′COX→RS—COR′

in which X is preferentially a halogenide in the compound R′CO—X.

(2) by modification agents that reversibly modify the cysteinyls of the present invention such as, for example, by heavy metals, in particular Zn²⁺, Cd²⁺ (Matts et al, 1991), mono-, dithio- and disulfide-compounds (e.g. aryl- and alkylmethanethiosulfonate, dithiopyridine, dithiomorpholine, dihydrolipoamide, Ellmann reagent, aldrothiol™ (Aldrich) (Rein et al, 1996), dithiocarbamates), or thiolation agents (e.g. gluthathion, N-Acetyl cysteine, cysteineamine). Dithiocarbamate comprise a broad class of molecules possessing an R₁R₂NC(S)SR₃ functional group, which gives them the ability to react with sulphydryl groups. Thiol containing compounds are preferentially used in a concentration of 0.1-50 mM, more preferentially in a concentration of 1-50 mM, and even more preferentially in a concentration of 10-50 mM;

(3) by the presence of modification agents that preserve the thiol status (stabilise), in particular antioxidantia, such as for example DTT, dihydroascorbate, vitamin s and derivates, mannitol, amino acids, peptides and derivates (e.g. histidine, ergothioneine, carnosine, methionine), gallates, hydroxyanisole, hydoxytoluene, hydroquinon, hydroxymethylphenol and their derivates in concentration range of 10 μM-10 mM, more preferentially in a concentration of 1-10 mM;

(4) by thiol stabilising conditions such as, for example, (i) cofactors as metal ions (Zn²⁺, Mg²⁺), ATP, (ii) pH control (e.g. for proteins in most cases pH −5 or pH is preferentially thiol pKa-2; e.g. for peptides purified by Reverse Phase Chromatography at pH −2).

Combinations of reversible protection as described in (1), (2), (3) and (4) may result in similarly pure and refolded HCV proteins. In effect, combination compounds can be used, such as, for example Z103 (Zn carnosine), preferentially in a concentration of 1-10 mM.

It should be clear that reversible protection also refers to, besides the modification groups or shielding described above, any cysteinyl protection method which may be reversed enzymatically or chemically, without disrupting the peptide backbone. In this respect, the present invention specifically refers to peptides prepared by classical chemical synthesis (see above), in which, for example, thioester bounds are cleaved by thioesterase, basic buffer conditions (Beekman et al., 1997) or by hydroxylamine treatment (Vingerhoeds et al, 1996).

Thiol containing HCV proteins can be purified, for example, on affinity chromatography resins which contain (1) a cleavable connector arm containing a disulfide bond (e.g. immobilised 5,5′ dithiobis(2-nitrobenzoic acid) (Jayabaskaran et al., 1987) and covalent chromatography on activated thiol-Sepharose 4B (Pharmacia)) or (2) a aminohexanoyl-4-aminophenylarsine as immobilised ligand. The latter affinity matrix has been used for the purifcation of proteins, which are subject to redox regulation and dithiol proteins that are targets for oxidative stress (Kalef et al., 1993).

Reversible protection may also be used to increase the solubilisation and extraction of peptides (Pomroy & Deber, 1998)

The reversible protection and thiol stabilizing compounds may be presented under a monomeric, polymeric or liposomic form.

The removal of the reversibly protection state of the cysteine residues can chemically or enzymatically accomplished by e.g.:

a reductant, in particular DTT, DTE, 2-mercaptoethanol, dithionite, SnCl₂, sodium borohydride, hydroxylamine, TCEP, in particular in a concentration of 1-200 mM, more preferentially in a concentration of 50-200 mM;

removal of the thiol stabilising conditions or agents by e.g. pH increase;

enzymes, in particular thioesterases, glutaredoxine, thioredoxine, in particular in a concentration of 0.01-5 μM, even more particular in a concentration of 0.1-5 μM.;

combinations of the above described chemical and/or enzymatical conditions.

The removal of the reversibly protection state of the cysteine residues can be carried out in vitro or in vivo, e.g. in a cell or in an individual.

It will be appreciated that after the second phase of the purification procedure, the cysteine residues may or may not be irreversibly blocked, or replaced by any reversible modification agent, as listed above.

A reductant according to the present invention is any agent which achieves reduction of the sulfur incysteine residues, e.g. “S—S” disulfide bridges, desulphonation of the cysteine residue (RS—SO₃⁻->RSH). An antioxidant is any reagent which preserves the thiol status or minimises “S—S” formation and/or exchanges. Reduction of the “S—S” disulfide bridges is a chemical reaction whereby the disulfides are reduced to thiol (—SH). The disulfide bridge breaking agents and methods disclosed in WO 96/04385 are hereby incorporated by reference in the present description. “S—S” Reduction can be obtained by (1) enzymatic cascade pathways or by (2) reducing compounds. Enzymes like thioredoxin, glutaredoxin are known to be involved in the in vivo reduction of disulfides and have also been shown to be effective in reducing “S—S” bridges in vitro. Disulfide bonds are rapidly cleaved by reduced thioredoxin at pH 7.0, with an apparent second order rate that is around 10⁴ times larger than the corresponding rate constant for the reaction with DTT. The reduction kinetic can be dramatically increased by preincubation the protein solution with 1 mM DTT or dihydrolipoamide (Holmgren, 1979).

Thiol compounds able to reduce protein disulfide bridges are for instance Dithiothreitol (DTT), Dithioerythritol (DTE), 3-mercaptoethanol, thiocarbamates, bis(2-mercaptoethyl)sulfone and N,N′-bis(mercaptoacetyl)hydrazine, and sodium-dithionite.

Reducing agents without thiol groups like ascorbate or stannous chloride (SnCl₂), which have been shown to be very useful in the reduction of disulfide bridges in monoclonal antibodies (Thakur et al., 1991), may also be used for the reduction of HCV proteins. In addition, changes in pH values may influence the redox status of HCV proteins. Sodium borohydride treatment has been shown to be effective for the reduction of disulfide bridges in peptides (Gailit, 1993). Tris(2-carboxyethyl)phosphine (TCEP) is able to reduce disulfides at low pH (Burns et al., 1991). Selenol catalyses the reduction of disulfide to thiols when DTT or sodium borohydride is used as reductant. Selenocysteamine, a commercially available diselenide, was used as precursor of the catalyst (Singh and Kats, 1995).

It is stressed again that the whole content, including all definitions of the documents cited above, are incorporated by reference in the present application. Hence, the above mentioned methods and compounds to modify the redox status of HCV proteins are all contemplated in the present invention.

Bio-Active Site

The present invention further pertains to HCV proteins containing a biologically active CXXC-motif. The terms “biologically active” and “oxido-reductase activity” as used herein contemplate a CXXC-site in a HCV peptide, or a functionally equivalent part thereof, with a reversible redox status, that has the ability to mediate various intra- and extracellular biochemical and biological functions, such as, for example, thiol/disulfide exchange reactions, binding of transition metals, lipid incorporation, and regulatory activities (e.g. control of gene transcription, regulation of signal transduction, including functioning as a cytokine, and the like, and control of the (de)thiolation status of proteins).

Structural or conformational changes effected by the cysteinyl redox status may be followed with biophysical methods, such as for example by spectrophotometry (absorbance, Circular Dichroism, Infrared, fluorescence, NMR) or with immunochemical methods (e.g. ELISA, EIA, and the like), which are based on the appearance or disappearance of epitopes. Sequences involved in the epitopes can be identified by Mass spectrometry (MS) and sequencing after cross-linking and affinity purification of the complex. The conformational or new detectable linear epitopes may result from folding processes on tertiary or quaternary structure level. Metal ion incorporation in the active site can be measured by radioactive decay measures or Atomic absorbance spectrometry.

The binding of the HCV proteins of the present invention to other molecules, such as for example receptors, carbohydrates, lipids, nucleic acids (see also above) can be studied by e.g. FACS, Biacore, immunological assays (Western blotting, EIA, ELISA, and the like), crosslinking and chromatographical methods (e.g. affinity-chromatography, gel filtration). Thioredoxin enzymatic activity of HCV proteins can be identified by studying the potential to reduce disulphide bridges according to the method as described by Holmgren et al. (1979). The effect of cofactors, such as DTT or dihydrolipoamide, can be verified in this method as well. Non-proteinaceous compounds (e.g. Ellmann reagent, aldrothiol) as well as proteins (e.g. aggregated insulin) can be taken as substrates.

The formation of mixed disulphides (see below), is an activity which is related to protein folding, and restoration of the active site. The formation of mixed disulphides can be demonstrated by reversible protection or irreversible blocking of the thiol groups before and after reductant treatment with different agents (e.g. DTT), such as described in “purification procedure”, followed by mass spectrometry analysis.

The pKa of the thiol groups in the —CXXC-containing protein is defined by treament with alkylation agents in function of the pH (titration). Differential protection and/or blocking of the residues and MS give information of the reaction initiating cysteinyl residue in the CXXC-site. Amino terminal amino acid sequencing can give information about the processing, cleavage products and domain structure of the HCV protein.

The tissue and intracellular distribution of these cleavage products are localized by immuno-histochemical methods.

Vaccine

The present invention also relates to a composition comprising a protein as defined above.

More particularly the present invention relates to a vaccine composition. The term “vaccine composition” relates to an immunogenic composition capable of eliciting protection against HCV, whether partial or complete. It therefore includes HCV peptides, proteins, polynucleotides, HCV-derived molecules or HCV-derived particles, as defined above. Protection against HCV refers in particular to humans, but refers also to non-human primates, trimera mouse (Zauberman et al., 1999), or other mammals.

The proteins of the present invention can be used as such, in a biotinylated form (as explained in WO 93/18054) and/or complexed to Neutralite Avidin (Molecular Probes Inc., Eugene, Oreg., USA). It should also be noted that “a vaccine composition” comprises, in addition to an active substance, a suitable excipient, diluent, carrier and/or adjuvant which, by themselves, do not induce the production of antibodies harmful to the individual receiving the composition nor do they elicit protection. Suitable carriers are typically large slowly metabolized macromolecules such as proteins, polysaccharides, polylactic acids, polyglycolic acids, polymeric amino acids, amino acid copolymers and inactive virus particles. Such carriers are well known to those skilled in the art. Preferred adjuvants to enhance effectiveness of the composition include, but are not limited to: colloidal iron hydroxide (Leibl et al., 1999), aluminium hydroxide, aluminium in combination with 3-O-deacylated monophosphoryl lipid A as described in WO 93/19780, aluminium phosphate as described in WO 93/24148, N-acetyl-muramyl-L-threonyl-D-isoglutamine as described in U.S. Pat. No. 4,606,918, N-acetyl-normuramyl-L-alanyl-D-isoglutamine, N-acetylmuramyl-L-alanyl-D-isoglutamyl-L-alanine2-(1′2′dipalmitoyl-sn-glycero-3-hydroxy-phosphoryloxy)ethylamine and RIBI (ImmunoChem Research Inc., Hamilton, Mont., USA) which contains monophosphoryl lipid A, detoxified endotoxin, trehalose-6,6-dimycolate, and cell wall skeleton (MPL+TDM+CWS) in a 2% squalene/Tween 80 emulsion. Any of the three components MPL, TDM or CWS may also be used alone or combined 2 by 2. Additionally, adjuvants such as Stimulon (Cambridge Bioscience, Worcester, Mass., USA) or SAF-1 (Syntex) may be used, as well as adjuvants such as combinations between QS21 and 3-de-O-acetylated monophosphoryl lipid A (WO94/00153), or MF-59 (Chiron), or poly[di(carboxylatophenoxy) phosphazene] based adjuvants (Virus Research Institute), or blockcopolymer based adjuvants such as Optivax (Vaxcel, Cythx) or inulin-based adjuvants, such as Algammulin and Gammalnulin (Anutech), Incomplete Freund's Adjuvant (IFA) or Gerbu preparations (Gerbu Biotechnik). It is to be understood that Complete Freund's Adjuvant (CFA) may be used for non-human applications and research purposes as well. “A vaccine composition” will further contain excipients and diluents, which are inherently non-toxic and non-therapeutic, such as water, saline, glycerol, ethanol, wetting or emulsifying agents, pH buffering substances, preservatives, and the like. The reversible modification of cysteinyl residues of the HCV peptides of the present invention, allows that these HCV peptides can be coupled covalently to a chemically activated carrier molecule, such as, for example, polymers or liposomes, or that the HCV peptide itself functions as carrier for binding other HCV-related or HCV non-related immunogenic proteins (mixed vaccines). HCV peptides linked to liposomes by a thioester have the advantage that the bonds are broken in vivo by host thioesterases, resulting in a slow antigen release and presentation. Incorporation or binding of the HCV peptide to the polymer or liposome can also be based on non-covalent interactions, exploiting affinity between the ligands.

Typically, a vaccine composition is prepared as an injectable, either as a liquid solution or suspension. Solid forms, suitable for solution on, or suspension in, liquid vehicles prior to injection may also be prepared. The preparation may also be emulsified or encapsulated in liposomes for enhancing adjuvant effect. The polypeptides may also be incorporated into Immune Stimulating Complexes together with saponins, for example Quil A (ISCOMS). Vaccine compositions comprise an immunologically effective amount of the polypeptides of the present invention, as well as any other of the above-mentioned components. “Immunologically effective amount” means that the administration of that amount to an individual, either in a single dose or as part of a series, is effective for prevention or treatment. This amount varies depending upon the health and physical condition of the individual to be treated, the taxonomic group of the individual to be treated (e.g. human, non-human primate, primate, etc.), the capacity of the individual's immune system to mount an effective immune response, the degree of protection desired, the formulation of the vaccine, the treating doctor's assessment, the strain of the infecting HCV and other relevant factors. It is expected that the amount will fall in a relatively broad range that can be determined through routine trials. Usually, the amount will vary from 0.01 to 1000 μg/dose, more particularly from 0.1 to 100 μg/dose. The vaccine compositions are conventionally administered parenterally, typically by injection, for example, subcutaneously or intramuscularly. Additional formulations suitable for other methods of administration include oral formulations and suppositories. Dosage treatment may be a single dose schedule or a multiple dose schedule. The vaccine may be administered in conjunction with other immunoregulatory agents.

DNA Vaccine

The intracellular environment of a host can provide the basis for the reversible redox-status of the HCV proteins of the present invention. In this regard, it should be clear that an HCV DNA vaccine composition comprises a plasmid vector comprising a polynucleotide sequence encoding an HCV protein as described above, operably linked to transcription regulatory elements. As used herein, a “plasmid vector” refers to a nucleic acid molecule capable of transporting another nucleic acid to which it has been linked. Preferred vectors are those capable of autonomous replication and/or expression of nucleic acids to which they have been linked. In general, but not limited to those, plasmid vectors are circular double stranded DNA loops which, in their vector form, are not bound to the chromosome. As used herein, a “polynucleotide sequence” refers to polynucleotides such as deoxyribonucleic acid (DNA), and, where appropriate, ribonucleic acid (RNA). The term should also be understood to include, as equivalents, analogs of either RNA or DNA made from nucleotide analogs, and single (sense or antisense) and double-stranded polynucleotides. As used herein, the term “transcription regulatory elements” refers to a nucleotide sequence which contains essential regulatory elements, such that upon introduction into a living vertebrate cell it is able to direct the cellular machinery to produce translation products encoded by the polynucleotide. The term “operably linked” refers to a juxtaposition wherein the components are configured so as to perform their usual function. Thus, transcription regulatory elements operably linked to a nucleotide sequence are capable of effecting the expression of said nucleotide sequence. Those skilled in the art can appreciate that different transcriptional promoters, terminators, carrier vectors or specific gene sequences may be used succesfully.

The instant invention pertains thus also to the use of an HCV protein as defined herein for prophylactically inducing immunity against HCV (prophylactic vaccine). It should be noted that a vaccine may also be useful for treatment of an individual as pointed-out above, in which case it is called a “therapeutic vaccine”.

It is clear from the above that the present invention also relates to the usage of a protein as defined above or a composition as defined above for the manufacture of an HCV vaccine composition. In particular, the present invention relates to the usage of a protein as defined herein for inducing immunity against HCV in chronic HCV carriers. More in particular, the present invention relates to the usage of a protein as defined herein for inducing immunity against HCV in chronic HCV carriers prior to, simultaneously to or after any other therapy, such as, for example, the well-known interferon therapy either or not in combination with the administration of small drugs treating HCV, such as, for example, ribavirin. Such composition may also be employed before or after liver transplantation, or after presumed infection, such as, for example, needle-stick injury. In addition, the present invention relates to a kit containing the HCV proteins of the present invention to detect HCV antibodies present in a biological sample.

The term “biological sample” as used herein, refers to a sample of tissue or fluid isolated from an individual, including but not limited to, for example, serum, plasma, lymph fluid, the external sections of the skin, respiratory intestinal, and genitourinary tracts, oocytes, tears, saliva, milk, blood cells, tumors, organs, gastric secretions, mucus, spinal cord fluid, external secretions such as, for example, excrement, urine, sperm, and the like.

Since the HCV proteins of the present invention are highly immunogenic, and stimulate both the humoral and cellular immune response, the present invention relates also to a kit for detecting HCV related T cell response, comprising the HCV protein of the instant invention. HCV T cell response can for example be measured as described in PCT/EP 94/03555 to Leroux-Roels et al. It should be stressed that the whole content, including all the definitions, of this document is incorporated by reference in the present application.

The present invention also relates to a composition as defined above which also comprises HCV core, E1, E2, P7, NS2, NS3, NS4A, NS4B, NS5A and/or NS5B protein, or parts thereof. E1, E2, and/or E1 E2 particles may, for example, be combined with T cell stimulating antigens, such as, for example, core, P7, NS3, NS4A, NS4B, NS5A and/or NS5B.

Moreover, the present invention also features the use of a protein as described above, or a composition as described above to detect antibodies against HCV proteins. As used herein, the term “to detect” refers to any assay known in the art suitable for detection. In particular, the term refers to any immunoassay as described in WO 96/13590.

Drug Screening

The invention provides methods for identifying compounds or agents which can be used to treat disorders characterized by (or associated with) HCV infection. These methods are also referred to herein as “drug screening assays” or “bioassays” and typically include the step of screening a candidate/test compound or agent for the ability to interact with (e.g., bind to) an HCV protein to modulate the interaction of an HCV protein and a target molecule, and/or to modulate HCV nucleic acid expression and/or HCV protein activity. Candidate/test compounds or agents which have one or more of these abilities can be used as drugs to treat disorders characterized by HCV infection, HCV nucleic acid expression and/or HCV protein activity. Candidate/test compounds such as small molecules, e.g., small organic molecules, and other drug candidates can be obtained, for example, from combinatorial and natural product libraries.

In one embodiment, the invention provides assays for screening candidate/test compounds which interact with (e.g., bind to) HCV protein, or any functionally equivalent part thereof. Typically, the assays are cell-free assays which include the steps of combining the HCV proteins of the present invention, its catalytic, i.e. oxido-reductase activity, or immunogenic fragments thereof, and a candidate/test compound, e.g., under conditions which allow for interaction of (e.g., binding of) the candidate/test compound to the HCV protein or portion thereof to form a complex, and detecting the formation of a complex, in which the ability of the candidate compound to interact with (e.g., bind to) the HCV protein or portion thereof is indicated by the presence of the candidate compound in the complex. Formation of complexes between the HCV protein and the candidate compound can be quantitated, for example, using standard immunoassays.

The HCV proteins, its catalytic or immunogenic fragments or oligopeptides thereof employed in such a test may be free in solution, affixed to a solid support, borne on a cell surface, or located intracellularly

In another embodiment, the invention provides screening assays to identify candidate/test compounds which modulate (e.g., stimulate or inhibit) the interaction (and most likely HCV protein activity as well) between an HCV protein and a molecule (target molecule) with which the HCV protein normally interacts, or antibodies which specifically recognize the HCV protein. Examples of such target molecules include proteins in the same signaling path as the HCV protein, e.g., proteins which may function upstream (including both stimulators and inhibitors of activity) or downstream of the HCV protein signaling pathway [Zn-fingers, protease activity, regulators of cysteine redox status].

Typically, the assays are cell-free assays which include the steps of combining an HCV protein of the present invention, its catalytic or immunogenic fragments thereof, an HCV protein target molecule (e.g., an HCV protein ligand) or a specific antibody and a candidate/test compound, e.g., under conditions wherein but for the presence of the candidate compound, the HCV protein or biologically active portion thereof interacts with (e.g., binds to) the target molecule or the antibody, and detecting the formation of a complex which includes the HCV protein and the target molecule or the antibody, or detecting the interaction/reaction of the HCV protein and the target molecule or antibody.

Detection of complex formation can include direct quantitation of the complex by, for example, measuring inductive effects of the HCV protein. A statistically significant change, such as a decrease, in the interaction of the HCV protein and target molecule (e.g., in the formation of a complex between the HCV protein and the target molecule) in the presence of a candidate compound (relative to what is detected in the absence of the candidate compound) is indicative of a modulation (e.g., stimulation or inhibition) of the interaction between the HCV protein and the target molecule. Modulation of the formation of complexes between the HCV protein and the target molecule can be quantitated using, for example, an immunoassay.

Therefore, the present invention contemplates a method for identifying compounds that modulate the interaction between binding partners in a complex, in which at least one of said binding partners is the HCV protein as defined above, and said method comprising:

(a) contacting a test compound with the complex, for a time sufficient to modulate the interaction in the complex; and thereafter

(b) monitoring said complex for changes in interactions, so that if a change in the interaction is detected, a compound that modulates the interaction is identified

In particular, the present invention contemplates the latter method in which at least one of the binding partners is selected from the group of:

(i) HCV-derived molecules, eg nucleic acids (promoters or enhancers) (HCV RNA packed in HCV particles) or proteins (structural or non-structural proteins)

(ii) Intracellular, host-derived molecules (modifiers of redox status of HCV peptides, (TRX, GRX, thioesterase, etc),)

(iii) Extracellular host-derived molecules (receptors, glucosamines, heparine)

It should be clear that modulators for interaction between binding partners in a complex, when identified by any of the herein described methods is contemplated in the invention.

To perform the above described drug screening assays, it is feasible to immobilize either HCV protein or its target molecule to facilitate separation of complexes from uncomplexed forms of one or both of the proteins, as well as to accommodate automation of the assay. Interaction (e.g., binding of) of HCV protein to a target molecule, in the presence and absence of a candidate compound, can be accomplished in any vessel suitable for containing the reactants. Examples of such vessels include microtitre plates, test tubes, and microcentrifuge tubes. In one embodiment, a fusion protein can be provided which adds a domain that allows the protein to be bound to a matrix. For example, HCV protein-His tagged can be adsorbed onto Ni-NTA microtitre plates (Paborsky et al., 1996), or HCV protein-ProtA fusions adsorbed to IgG, which are then combined with the cell lysates (e.g. (35)S-labeled) and the candidate compound, and the mixture incubated under conditions conducive to complex formation (e.g., at physiological conditions for salt and pH). Following incubation, the plates are washed to remove any unbound label, and the matrix immobilized and radiolabel determined directly, or in the supernatant after the complexes are dissociated. Alternatively, the complexes can be dissociated from the matrix, separated by SDS-PAGE, and the level of HCV protein-binding protein found in the bead fraction quantitated from the gel using standard electrophoretic techniques.

Other techniques for immobilizing protein on matrices can also be used in the drug screening assays of the invention. For example, either HCV protein or its target molecule can be immobilized utilizing conjugation of biotin and streptavidin. Biotinylated HCV protein molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with HCV protein but which do not interfere with binding of the protein to its target molecule can be derivatized to the wells of the plate, and HCV protein trapped in the wells by antibody conjugation. As described above, preparations of a HCV protein-binding protein and a candidate compound are incubated in the HCV protein-presenting wells of the plate, and the amount of complex trapped in the well can be quantitated. Methods for detecting such complexes, in addition to those described above for the GST-immobilized complexes, include immunodetection of complexes using antibodies reactive with the HCV protein target molecule, or which are reactive with HCV protein and compete with the target molecule; as well as enzyme-linked assays which rely on detecting an enzymatic activity associated with the target molecule.

Another technique for drug screening which provides for high throughput screening of compounds having suitable binding affinity to the HCV protein is described in detail in “Determination of Amino Acid Sequence Antigenicity” by Geysen H N, WO Application 84/03564, published on 13/09/84, and incorporated herein by reference. In summary, large numbers of different small peptide test compounds are synthesized on a solid substrate, such as plastic pins or some other surface. The protein test compounds are reacted with fragments of HCV protein and washed. Bound HCV protein is then detected by methods well known in the art. Purified HCV protein can also be coated directly onto plates for use in the aforementioned drug screening techniques. Alternatively, non-neutralizing antibodies can be used to capture the peptide and immobilize it on a solid support.

This invention also contemplates the use of competitive drug screening assays in which neutralizing antibodies capable of binding HCV protein specifically compete with a test compound for binding HCV protein. In this manner, the antibodies can be used to detect the presence of any protein which shares one or more antigenic determinants with HCV protein.

In yet another embodiment, the invention provides a method for identifying a compound (e.g., a screening assay) capable of use in the treatment of a disorder characterized by (or associated with) HCV infection, HCV nucleic acid expression or HCV protein activity. This method typically includes the step of assaying the ability of the compound or agent to modulate the expression of the HCV nucleic acid or the activity of the HCV protein thereby identifying a compound for treating a disorder characterized by HCV infection, HCV nucleic acid expression or HCV protein activity.

Modulators of HCV infection, HCV protein activity and/or HCV nucleic acid expression identified according to these drug screening assays can be used to treat, for example, HCV infection or disorders related to HCV infection.

These methods of treatment include the steps of administering the modulators of HCV protein activity and/or HCV nucleic acid expression, e.g., in a pharmaceutical composition as described above, to a subject in need of such treatment, e.g., a subject with an HCV infection.

The tissue or cell specificity of the drug may be enhanced by using the drug targeting methods (see Davis, 1997) or intracellular immunisation. Liver targeting tools are for example biluribin coupled drugs (Kramer et al. 1992), asialoglycoprotein receptor or lipoprotein mediated transfer of drugs (Vingerhoeds et al. 1996). Drugs may even intracellularly targeted with cell organel targeting of DNA expressed molecule via cell organel specific targeting tags (Persic et al 1997).

Methods for assaying the ability of the compound or agent to modulate the infection of HCV, the expression of the HCV nucleic acid or activity of the HCV protein are typically cell-based assays. However, HCV infected animals are also contemplated herein. For example, HCV infected or transfected cells which are sensitive to reductants or oxidants, or which transduce signals via a pathway involving HCV protein can be induced to overexpress an HCV protein in the presence and absence of a candidate compound.

Candidate compounds which produce a statistically significant change in HCV protein—dependent responses (either stimulation or inhibition) can be identified.

In one embodiment, infection of target cells by HCV, expression of the HCV nucleic acid or the oxido-reductase activity of an HCV protein is modulated in cells and the effects of candidate compounds on the readout of interest (such as rate of infection, cell proliferation or differentiation, or oxido-reductase activity) are measured. For example, the transition rate from the thiolated form to the S-conjugated, i.e. S—S bridge, form can be assayed. For example, the expression of genes which are up- or down-regulated in response to an HCV protein-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of HCV protein or HCV protein target molecules can also be measured, for example, by immunoblotting.

Therefore the present invention pertains to a bioassay for identifying compounds that modulate the oxido-reductase activity of HCV proteins as defined above, said bioassay comprising:

(a) exposing cells expressing HCV proteins, or any functionally equivalent part thereof, as defined above to at least one compound whose ability to modulate the oxido-reductase activity of said proteins is sought to be determined; and thereafter

(b) monitoring said proteins for changes in oxido-reductase activity.

The reversibly protected HCV peptide may be used for diagnostic coupling purposes in, for example, an oligomerised state as (1) chemical polyantigen preparations (the E1s coupled antigens are not necessary HCV related and may thus be used for multi-disease screening); (2) targets for immobilisation and immunodetection (e.g. biotinylation, fluorescence) or (3) for antibody conjugation, which in turn may result in supramolecular antibodies (e.g. Antibodies on virus-like particles). The labelling and antibody conjugation result in an increase of sensitivity due to the amplification step by oligomerisation of the protein.

It has to be mentioned that any reactive group on the peptide (sugars, amino, carboxyl, thiol, histidine, and the like) may be exploited for the coupling or conjugation. The reversible protected group can be used to enhance the specificity of reaction and the thiol reactivity can be exploited in a later step/phase of conjugation after deprotection.

Finally, the present invention relates to an immunoassay for detecting HCV antibody, which immunoassay comprises: (1) providing the purified HCV protein as defined herein, or a functional equivalent thereof, (2) incubating a biological sample with said HCV protein under conditions that allow the formation of antibody-antigen complex, (3) determining whether said antibody-antigen complex comprising said HCV protein is formed.

The present invention will now be illustrated by reference to the following examples which set forth particularly advantageous embodiments. However, it should be noted that these embodiments are merely illustrative and can not be construed as to restrict the invention in any way.

### EXAMPLES

### Example 1

### Determination of the thiol-disulphide Status in Vaccinia Expressed E1s

HCV E1s protein (amino acids 192-326) was expressed and purified from Vero cells using recombinant vaccinia virus pv-HCV11A according to the protocol as described in Maertens et al. (PCT/EP 95/03031), except that the blocking of the thiol groups was done with Iodo-acetamide and N-ethylmaleimide (NEM) during the lysis and after the reduction with DTT, respectively. Thus, blocking free thiols with IAA (Iodoacetamide) in the lysis buffer, and alkylation with NEM after the reduction step with DTT.

The purified E1s was concentrated by ultrafiltration (Centricon 10, Millipore), deglycosylated with N-glycosidase F (PGNase F; Boehringer Mannheim) as described by the manufacturer, after which the E1s was loaded on a 15% polyacrylamide minigel. SDS-PAGE was performed as described by Laemmli. The protein bands were cut in the ca. 18 kDalton region after size separation and staining.

Proteins were cleaved by in situ trypsinolysis, and the resulting peptide digest was analysed by mass spectroscopy (MS; MALDI-TOFF) to determine the derivatisation state of the different cysteine-residues.

The MS results show for the cysteines in the CXXC-motif that:

(1) in ca. 10% of the CXXC-motifs, both cysteines were present as IAA derivatised products;

(2) in ca. 30% of the CXXC-motifs, one cysteine was blocked with IAA, the other cysteine was present as NEM-derivatised product;

(3) in ca. 60% of the CXXC-motifs, both cysteines were retrieved as NEM-derivatised product.

These data show surprisingly that

(1) either the two cysteines were present in a fully reduced (“thiol”) status; and

(2) either one of the cysteines was involved in a mixed disulfide bridge and the second cysteine was present as free thiol (intermediate form); and

(3) that both cysteines were present in the oxidised form (disulfide bridge).

Although the experiment is performed with an HCV peptide, ie derived from an infectious pathogen, these three forms correlate well with the different oxidation status which has been described for the —CXXC-motif in the TRX superfamily, and correspond with the activity pattern described for the thiol oxidoreductases, ie molecules involved in regulating oxido-reduction environment in the cell (Rietsch & Beckwith, 1998; Loferer & Hennecke, 1994; Aslund & Beckwith, 1999; Huppa & Ploegh, 1999).

Since the cysteines in the active site of thioredoxin are oxidised and the disulfide bridge in the substrate is reduced, the excess of the oxidised form (60%) is in agreement with thioredoxin activity. Surprisingly, these results tend to indicate that E1s is involved in an (auto)folding mechanism, that is dependent on on the intracellular oxidative status for the regeneration of the active site to the reduced form. Therefore, the —S—S— protein based aggregate consisting of E1s, vaccinia and host proteins can be diminished by interfering at the level of protein folding or by addition of compounds in the culture media which interfere/influence the intracellular redox status of cysteines.

### Example 2

### Purification of Yeast E1s-His After Reversible Modification of Cysteines

Saccharomyces cerevesiae (yeast) cells producing his-tagged HCV E1s were harvested by microfiltration and centrifugation. The cell pellets are resuspended in 5 volumes lysis buffer (50 mM phosphate, 6M Guanidinium-HCl, pH 7.4 (=buffer A)) and solid Na₂SO₃, Na₂S₄O₆ are added to the solution till a final concentration of 160 mM and 65 mM, respectively. Cu²⁺ (100 mM stock solution in NH₃) is added as catalysator till a concentration of 100 μM and the solution is incubated overnight at room temperature. The lysate is stored at −70° C. and cleared by centrifugation (JA 20 rotor, 27 kg at 4° C.) after the freeze-thaw cycle.

Imidazole and Empigen (Albright & Wilson, UK) are added to the supernatant, respectively, till a final concentration of 20 mM and 1% (w/v) and the sample is applied on a Ni-IDA Sepharose FF column (Pharmacia) after dilution with the equilibration buffer (Buffer A, 20 mM Imidazole, 1% Empigen).

The resin was washed with the equilibration buffer till the absorbance at 280 nm reaches baseline level and the bound proteins are eluted by applying an imidazole step gradient. SDS-PAGE and Western blot analysis show that >90% pure E1s-His protein is retrieved in the 200 mM Imidazole elution pool after sulfitolysis under denaturing conditions and IMAC (FIG. 6B).

Sulphonated HCV E1s is desulphonated by addition of DTT to restore the thiol status and allow the formation of intra-and inter molecular disulphide bridge.

### Example 3

### Purification and Immunological Reactivity of E. coli NS3 Fusion Protein

E. coli cells producing the mTNF(His)₆NS3 B9 fusion protein were harvested and the cells were resuspended in buffer A (see Example 2). Sulfonation, sample preparation and metal chromatography run on Ni-IDA Sepharose FF (Pharmacia) were done as described for yeast (Example 2). The mTNF(His)₆ NS3 b9 fusion protein was retrieved in the 200 mM Imidazole elution pool. Coomassie staining of SDS-PAGE gels and Western blot showed that the HCV fusion protein is >90% pure after sulfitolysis and IMAC (see FIG. 6A).

The immune reactivity of the fusion protein was checked by ELISA with HCV positive human sera. The purified fusion protein was reduced with 200 mM DTT and the protein was desalted to 35 mM acetate, 6 M ureum, pH 4 on a Sephadex G25 column (Pharmacia). The effect of anti-oxidants and reversible protecting agents (dithiocarbamate, GSH, cysteine) on the NS3 fusion protein reactivity was verified by adding these agents either before freezing at −70° or by adding these compounds during the dilution in the ELISA coating buffer.

NS3 fusion protein coated in the presence of 10 mM or 200 mM DTT were included as positive and negative control, respectively. Sera (17790, 17832) are difficult detectable sera (HCV NS3 converting sera) and sera (17826, 17838) are easily detectable HCV positive sera. HCV sera which are difficult to detect are (1) sera which react not or minimal with other HCV antigens (NS3 onlies) or (2) sera which react with NS3-epitopes which are only presented and recognized by antibodies after treatment of sulfonated NS3 b9 with 200 mM DTT. In contrast, for easy detectable sera a treatment with 10 mM DTT of sulfonated NS3b9 is sufficient for restoring the immunological reactivity. The ELISA results are given in FIGS. 5A and 5B.

The results show that the disponibility of the epitopes is strongly dependent on the thiol redox state, i.e. the difficult HCV sera are only detected either (1) after reduction of the NS3 with 200 mM DTT in the coating buffer or (2) by incubation of the sample diluent in the presence of 10 mM or 3 mM DTT, provided that the NS3 sample is diluted with thiol containing antioxidantia and/or reversible protection agents. The restoration of the immunological reactivity was more pronounced with dithiocarbamates than with gluthathion, cystein or thiophenecarboxylic acid (TPCB) or thiodiethyleneglycol (TEG). Gluthathion was in turn superior to Cystein or other tested mono-SH (TEG, TPCB) products. The best ELISA signals were obtained for NS3B9 fusion protein, which was incubated at −70° C. and diluted in the presence of thiol stabilising and reversible protective agents.

The need of the DTT reduction step to restore the immune reactivity after the addition of thiol containing compounds showed the formation of mixed disulfide bridges between the thiol agents and the cysteine residues of NS3 b9 fusion protein. The addition of thiol compounds have inhibited the reformation of the very stable intramolecular disulphide bond, that only could be reduced with 200 mM DTT. This mixed disulfide bridge status resembles the in vivo thiolation of proteins, which is known to be a regulator biological activity and is with minimal energy input transferred (enzymatically or by a ‘S—S’ reductant) to the reduced status.

### Example 4

### Mapping of Monoclonal Antibodies Against an E1 Epitope Overlapping with the Cysteine Residues from the CXXC Site

Ten monoclonal antibodies, directed against E1, were identified which recognize the N-terminal region of E1. These monoclonals were characterized regarding their minimal epitope.

In order to do so two peptides were synthesized and reactivity of each monoclonal towards these peptides was analyzed by assessing competition. Recombinant E1 was adsorbed to microtiterplates and the monoclonal antibody was allowed to react in the presence of an excess of the peptide. Based on these results the ten monoclonal antibodies can be split in two groups (Table 1). For the first group the minimal epitope is aa 209-227, especially the lack of reactivity with a peptide not containing the aminoacids 225-227 proves that these monoclonals cover an epitope overlapping with the thioredoxine-like site, more specifically with the first cysteine of this site. The minimal epitope of the monoclonals of group 2 does not reach into the thioredoxine-like site. These results are summarized in Table 1.

TABLE 1
minimal epitope delineation of
monoclonal antibodies directed against E1

| | | | |
|---|---|---|---|
| Sequence | aa region | IGP* | result |

E1 monoclonal antibodies group 1: IGH 198, 199 and 200

| | | | |
|---|---|---|---|
| NDCPNSSIVYEAHDAILHTP (*) | 205-22 | 263 | no competition |
| Bio-GG-SNSSIVYEAADMIMHTPGCV (**) | 208-227 | 436 | competition |
| (*) (SEQ ID NO 3) | | | |
| (**) (SEQ ID NO 4) | | | |

E1 monoclonal antibodies groupe 2:
IGH 201, 202, 203, 204, 205, 206 and 208

| | | | |
|---|---|---|---|
| NDCPNSSIVYEAHDAILHTP (*) | 205-224 | 263 | competition |
| Bio-GG-SNSSIVYEAADMIMHTPGCV (**) | 208-227 | 436 | competition |
| (*) (SEQ ID NO 3) | | | |
| (**) (SEQ ID NO 4) | | | |

The minimal epitope for each group is underlined
*IGP refers to the peptide code number
note 1
Also monoclonals are available recognizing an epitope in the C-terminal part of E1 (IGH 207, 209 and 210, aa 307-326; see PCT/EP99/02154). These monoclonals may be used as controls since they recognize a region which is not at all in the neighbourhood of the thioredoxine-like site.
note 2
IGH 198 = 23C12
IGH 199 = 15B5
IGH 200 = 25CF3
IGH 201 = 11B7
IGH 202 = 3F3
IGH 203 = 15G6
IGH 204 = 8A8
IGH 205 = 3H2
IGH 206 = 7C4
IGH 207 = 14H11
IGH 208 = 5C6
IGH 209 = 5E1
IGH 210 = 7D2

Thus, monoclonals are available which can be used as tools to determine changes in the biological activity and/or conformation of peptides of the present invention.

### Example 5

### HCV E1s Purification After Reversible Modification of Cys-Residues

Vaccinia RK13 cells were lysed as described in Maertens et al. (PCT EP95/03031), but solid sodium tetrathionate was added to the lysate up to 65 mM instead of the irreversible thiol blocking agent N-ethylmaleimide (NEM). The lysate was incubated overnight at 4° C. and the purification steps (Lentil lectin (LCA) chromatography, the concentration of the LCA eluate and reduction with DTT) were performed as described in Maertens et al. (PCT EP95/03031). The concentrate was split in 2 and was either sulfonated overnight at 4° C. by Na₂S₄O₆ or irreversible blocked with N-ethyl-maleimide (NEM) as reference material. The sulfonated as well as the NEM-treated E1s were applied on Superdex G200 (Pharmacia) in the presence of Empigen (see FIG. 1) and the E1s peak was analysed by SDS-PAGE and Western blot.

The chromatogram overlays as well as the ELISA profiles show that the irreversible protected and sulfonated E1s product behave analogously on SEC in the presence of Empigen. SDS-PAGE and silverstaining show a similar purity degree of the 2 products.

### Example 6

### HCV E1s Purification Under non-Denaturing Conditions After Lysis in the Presence of Thiol Stabilising Agents

Vaccinia infected RK13 cells were lysed as described in Maertens et al. (PCT EP95/03031), but ascorbate (1 mM) was added to the lysate as thiol stabiliser instead of NEM. The sample was applied on the LCA resin and the LCA eluate was acidified till pH 5.5 with 1M acetic acid.

The acidified eluate was concentrated, after which the pH was adjusted to 7.2 and treated with DTT as described in Maertens et al. (PCT EP95/03031).

The reduced protein solution was split and treated as follows: either (1) acidified to pH 6 (thiol stabilising conditions) or (2) sulfonated with sodium tetrathionate (reversibly protected) or (3) treated with NEM.bio (irreversible blocking). The SEC of the acidified (pH 6) sample was also performed at pH 6.0. The other 2 samples were separated on Superdex G200 in the presence of Empigen as decribed in Maertens et al. (PCT EP95/03031). The elution fractions were analysed by ELISA, by SDS-PAGE and Western blotting.

The material, prepared as described in Maertens et al. (PCT EP95/03031) is included as reference material for the SEC.

Fraction analysis shows that pure E1s is recovered for the different conditions (FIG. 3A.1 and FIG. 3A.2). The higher apparent Mr of NEM.bio E1s materials is probably caused by the insertion of voluminous blocking group on E1s.

FIG. 3B shows a Western blot of E1s pools, obtained by different procedures as described in Examples 5 and 6.

Examples 5 and 6 illustrate that pure E1s is obtained under non-denaturing conditions by (1) using reversible modification agents or (2) running the chromatography under thiol stabilising conditions (antioxidant, low pH).

### Example 7

### Processing of Vero E1s and Cleavage Analogy with Growth Factors, Such as Thioredoxine

Vaccinia infected Vero cells were lysed as described in Maertens et al. (PCT EP95/03031), but Iodoacetamide (IAA) was added as irreversible blocking agent and aprotinin was added after an overnight incubation at 4° C.

Chromatography on LCA resin (Pharmacia), reduction with DTT and gel filtrations were performed as described, except that IM was used as irreversible blocking agent instead of NEM. The E1s pool was analyzed by silver staining and Western blotting.

Western blot analysis of the semi purified product showed besides the quartet band in the region 27-32 kDa also an E1s band with an Mr of about 18 kDa. The bands were characterized by NH₂-terminal amino acid sequencing.

Main signal sequence of the different bands of the quartet:

Y E V R ? V S G

(amino-terminus of correctly processed E1s)

Sequence of E1s degradation product:

? ? V A L T P T L A A

This degradation product results from a specific cleavage at the carboxy-terminus after Arg 237, which is localized upstream of the CVPC-site. The first and second residue are not identified, because the cysteine and tryptophan amino acids are destroyed by the Edman sequencing method.

Surprisingly, no other degradation products were retrieved although other basic residues and even dibasic sequences are present in E1s. This specific cleavage pattern corresponds with a E1s domain structure, which has been described for the processing of growth factors, such as thioredoxine, which cleavage has resulted in the formation of ECEF (Balcewicz-Sablinska, et al., 1991; Newman et al., 1994).

### Example 8

### Titration of the pKa of the Cysteines in the E1s CVPC-Site

In order to establish the sequence of reaction steps, ie which cysteine of the C₁VPC₂-site reacts first, the pKa of these cysteines is titrated. The pKa of the cysteines in the C₁VPC₂-site of E1s is determined by modification of the cysteines in E1s or synthetic peptides in function of the pH.

The modification is performed by treatment with IAA at the preset pH, whereafter the sample is loaded on RPC after lowering the pH to 2 with Trifluotroacetic acid (TFA).

In order to determine the most reactive cysteine, the excess of IAA reagent is removed by RPC. The non-reacted thiol-groups are modified by raising the pH after addition of ethyleneimine (E1) or Bromo-ethanolamine (BEA).

The treatment with E1 or BEA results in the introduction of a lysine mimicking cysteine adduct, which creates a supplementary trypsinolysis site. This supplementary site allows the identification of most reactive cysteine in the —C₁VPC₂-site via peptide fingerprinting and MS (see also Example 1).

### REFERENCES

Atherton and Shepard in “Solid phase peptide synthesis” IRL Press, Oxford, 1989.

Aslund, F. and Beckwith, J. (1999) Bridge over troubled waters: sensing stress by disulfide bond formation. Cell 96: 751-753.

Balcewicz-Sablinska, M, Wollman, E., Gorti, R & Silberstein, D., Human eosinophil cytotoxicity-enhancing factor. Multiple forms synthesised by U937 cells and their relationship to thioredoxin/adult T cell leukemia-derived factor. J. Immunol. 147, 2170-2174,1991.

Beekman, N., Schaaper, W., Tesser, G., Dalsgaard, K., Kamstrup, S., Langeveld, J., Boshuizen, R.& Meloen, R., Synthetic peptide vaccines: palmitoylation of peptide antigens by a thioester bond increases immunogenicity. J.Peptide Res., 50, 357-364,1997.

Burns, J., Butler, J. & Whitesides, G., Selective reduction of disulfides by tris(2-carboxyethyl)phosphine. J.Org. Chem. 56, 2648-2650 (1991).

Carreras, C.,& Santi, D., Engineering of modular polyketide synthases to produce novel polyketides. Curr. Opion in Biotech., 9, 403-411 (1998).

Darbre, A., Practical protein Chemistry: A handbook. AWhiley-interscience publication. Ed. J. Whiley & Sons Ltd., 1986

Das, A. K., Dasgupta, B., Bhattacharya, R. Basu. J. (1997) Purification and biochemical characterisation of a protein-palmitoyl acyltransferase from human erythrocytes. J. Biol. Chem. 272:11021-11025.

Davis, S. S. (1997) Biomedical applications of nanotechnology—implications for drug targeting and gene therapy. Tibtech 15: 217-223.

Deleersnyder V., Pillez A., Wychowski C., Blight K., Xu J., Hahn Y. S., Rice C. M., Dubuisson J. Formation of native hepatitis C virus glycoprotein complexes. J. Virol. 1997: 71: 697-704.

Diepolder H M, Zachoval R, Hoffmann R M, Wierenga E A, Santantonio T, Jung M C, Eichenlaub D, Pape G R. Possible mechanism involving T-lymphocyte response to non-structural protein 3 in viral clearance in acute hepatitis C virus infection. Lancet 1995: 346:1006-1007.

Diepolder H M, Gerlach J T, Zachoval R, Hoffmann R M, Jung M C, Wierenga E A, Scholz S, Santantonio T, Houghton M, Southwood S, Sette A, Pape G R. Immunodominant CD4+ T-cell epitope within nonstructural protein 3 in acute hepatitis C virus infection. J. Virol., 1997: 71: 6011-6019.

Dietrich, C., Boshemen, O, Scharf, K., Schmitt, L.& Tampe, R., Functional immobilisation of a DNA-binding protein at a membrane interface via a histidine tag and synthetic chelator lipids. Biochemistry, 35, 1100-1105 (1996),

Dietrich, C., Schmitt, L.& Tampe, R., Molecular organisation of histidine-tagged bimolecules at self assembled lipid interfaces using a new class of chelator lipids. PNAS, 92, 9014-9018, 1995)

Fancy, D. A., Melcher, K., Johnston, S. T. and Kodadek, T. New chemistry for the study of multiprotein complexes: the six-histidine tag as a receptor for a protein crosslinking reagent. Chem Biol (1996) 3: 551-559.

Fernandes, A. & Gregoriadis, G., Polysialylated asparaginase: preparation, activity and pharmacokinetics. Biochem. Biophys. Acta, 1341, 26-34, 1997

Gailit, J. Restoring free sulfydryl groups in synthetic peptides. Anal. Biochem.,214,334-335 (1993).

Hermanson, G. T. in Bioconjugate Techniques (1996) Part I section 1.43 and section 2.2.1, Academic Press San Diego Calif., USA.

Holmgren, A., Thioredoxin catalyzes the reduction of insulin disulfides bydithiothreitol and dihydrolipoamide. J. Biol. Chem., 254, 9627-9632 (1979).

Houbenweyl in “Methode der Organischen Chemie” edited by E. Wunsch, vol 15-I et II. Thieme, Stuttgart (1974).

Houghton M. Immunity to HCV: The case for vaccine development. 4th International meeting on hepatitis C Virus and related viruses. Sattelite Symposium: New appraoch to prevention and therapy of HCV infection. March 7, 1997, Kyoto, Japan.

Huang H., Rabenstein, D. L. (1999) A cleavage cocktail for methionine-containing peptides. J.Peptide. Res. 53: 548-553. Reagent H, 10 h, in situ oxidation of cysteine, disulphide forms of peptides

Huppa, J & Ploegh, H., The eS-Sence of —SH in the ER. Cell, 92, 145-148 (1999).

Jayasbaskaran, J., Davison, P. & Paulus, H., Facile preparation and some applications of an affinity matrix with a cleavable connector arm containing a disulfide bond. Prep. Biochem., 17,121-141 (1987).

Jayasena, S. D. (1999) Aptamers: an emerging class of molecules that rival antibodies in diagnostics. Clin. Chem. 45:1628-1650.

Kalef, E, Walfish, P.& Gitler C., Arsenical based affinity chromatography of vicinal dithiol-containing proteins: Purification of L1210 Leukemia cytoplasmatic proteins and the recombinant rat c-erb Aβ₁T₃ receptor. Anal. Biochem.,212,325-334(1993).

Kramer, W., Wess, G., Schubert, G., Bickel, M., Girbig, F., Gutjahr, U., Kowalewski, S., Baring-haus, KH., Ehnsen, A., Glombik, H., Mullner, S., Neckermann, G., Schulz, S. & Petzinger, E., (1992) Liver-specific drug targeting by coupling to bile acids. J. Biol. Chem. 267: 18598-18604.

Kumar, N, Kella, D & Kinsella, J., Anomalous effect of denaturants on sulfitolysis of protein disulfide bonds. Int. J. Peptide Protein Res., 28, 586-592, (1986).

Kumar, N, Kella, D.& Kinsella, J., A method for the controlled cleavage of disulfide bonds in proteins in the absence of denaturants. J. Biochem. Biophys. Meth., 11, 253-261,1985.

Leroux-Roels G, Esquivel C A, DeLeys R, Stuyver L, Elewaut A, Philippe J, Desombere I, Paradijs J, Maertens G Lymphoproliferative responses to hepatitis C virus core, E1, E2, and NS3 in patients with chronic hepatitis C infection treated with interferon alfa. Hepatology 1996: 23: 8-16.

Leibl H, Tomasits R, Bruhl P, Kerschbaum A, Eibl M M, Mannhalter J W (1999) Humoral and cellular immunity induced by antigens adjuvanted with colloidal iron hydroxide. Vaccine 17:1017-23

Loferer, H.& Hennecke, H., Protein disulphide oxidoreductases in bacteria. TIBS, 19, 169-171,(1994).

Maertens G. and Stuyver L. Genotypes and genetic variation of hepatitis C virus. In: The molecular medicine of viral hepatitis. Ed: Harrison T. J. and Zuckerman A. J. 1997

Maertens G., Depla E., Ducatteeuw A., Vandeponseele P., Bosman F., Venneman A., de Martynoff G., Stuyver L., Dekeyser F., Vandeperre B., Zrein M. And Buyse M.-A. Hepatology 1997: 26: 186A.

Major M. E. and Feinstone S. M. The molecular virology of hepatitis C. Hepatology 1997: 25:1527-1538.

Mossner, E., Huber-Wunderlich, M.& Glockshuber, R., Characterisation of E. coli thioredoxin variants mimicking the active sites of other thiol/disulfide oxidoreductases. Prot. Science, 7, 1233-1244,1998.

Matts, R., Schatz, J., Hurst, R & Kagen, R., Toxic heavy metal ions activate the heme-regulated eucaryotic initiation factor 2 kinase by inhibiting the capacity of hemin-supplemented reticulocyte lysates to reduce disulfide bonds. J. Biol. Chem., 6, 12695-12702 (1991).

Nakamura, H., Nakamura, K. and Yodoi, J. (1997) Redox regulation of cellular activation. Annu. Rev. Immunol. 15: 351-369 Newman, G W, Balcewicz-Sablinska, M K, Guarnaccia, J, Remold, H & Silberstein, D, Opposing regulatoty effects of thioredoxin and eosinophil cytotoxicity-enhancing factor on the development of human immunodeficiency virus 1. J. Exp. Med., 180, 359-363, 1994

Nielsen, P., Egholm, M, Berg, R., and Buchardt, 0. (1991) Science 254: 1497-1500.

Nielsen, P., Egholm, M, Berg, R., and Buchardt, 0. (1993) Nucl. Acids Res. 21: 197-200.

Paborsky, et al. (1996) Anal. Biochem. 234: 60-65.

Persic, L., Righi, M., Roberts, A., Hoogenboom, HR, Cattaneo, A & Bradbury, A., Targeting vectors for intracellular immunisation. Gene, 187, 1-8,1997.

Pinter, A., Kopelman, R., Li, Z., Kayman, S. C. and Sanders, D. A. (1997) Localization of the labile disulfide bond between SU and TM of the murine Leukemia virus envelope protein complex to a highly conserved CWLC motif in SU that resembles the active-site of the thiol-disulfide exchange enzymes. J. Virology 71: 8073-8077.

Pomroy, N & Deber, C., Solubilisation of hydrophobic peptides by reversible cysteine PEGylation. Biochem. & Biophys. Res. Commun., 245, 618-621 (1998).

Prinz, W., Aslund, F, Holmgren, A.& Beckwith, J., The role of thioredoxin and glutaredoxin pathways in reducing protein disulfide bonds in E. coli cytoplasm. J. Biol. Chem., 272, 15661-15667, 1997.

Rehermann B, Chang K M, McHutchinson J, Kokka R, Houghton M, Rice C M, Chisari F V. Differential cytotoxic T-lymphocyte responsiveness to the hepatitis B and C viruses in chronically infected patients. J Virol 1996 70: 7092-7102.

Rehermann B, Takaki A, Liebetrau A, Luda S, Seifert U, Salha K, Manns M, Wiese M. Characterization of the cytotoxic and helper T cell response in patients 18 years after a single source outbreak of HCV infection. Hepatology, 1997:26: 406A

Rein, A., Ott, D., Mirro, J., Arthur, L, Rice, W.& Henderson, L., Inactivation of Murine leukemia virus by compounds that react with the Zn-finger in viral nucleocapsid protein. J. Virol., 70, 4966-4972,1996.

Rietsch, A. & Beckwith, J., The genetics of disulfide bond metabolism. Annu. Rev. Genet., 32, 163-184(1998).

Sambrook, J., Fritsch, E. F. and Maniatis, T. (1989) Molecular Cloning, a laboratory manual, second edition. Cold Spring Harbor University Press, Cold Spring Harbor, N.Y. USA

Singh, R. & Kats, L., Catalysis of reduction of disulfide by selenol. Anal. Biochem., 232,86-91 (1995).

Southworth, M., Amaya, K., Evans, T., Xu M.& Perler, F., Purification of proteins to eiteher the amino or carboxy terminus of the Mycobacterium xenopii gyrase A intein. Biotechniques, 27, 110-120,1999.

Thakur, M., Defulvio, J., Richard, M & Park, C., Technetium-99m labelled monoclonal antibodies: evaluation of reducing agents. Nuc. Med. Biol., 18, 227-2333(1991)

Thomas, J. A., Poland, B. and Honzatko, R. (1995) Protein sulfhydryls and their role in the antioxidant function of protein S-thiolation. Arch. Biochem. Biophys. 319: 1-9.

van Doom L J, Kleter B, Pike I, Quint W. Analysis of hepatitis C virus isolates by serotyping and genotyping. J Clin Microbiol 1996; 34:1784-1787.

Villa E., Buttafoco P., Grottola A., Scarcelli A., Giannini F., Manerti F. Neutrtalizing antibodies against HCV: liver transplant as an experimental model. J. Hepatol. 1998: 28:

Vingerhoeds, M., Haisma, H., Belliot, S., Smit, R., Crommelin, D. & Storm, G, Immunoliposomes as enzyme-carriers (immunoenzyzomes) for antibody-directed enzyme prodrug therapy (ADEPT): optimization of prodrug activating capacity. Pharm.Res.,13,603-610 (1996).

Weiner A J, Erickson A L, Kansopon J, Crawford K, Muchmore E, Houghton M, Walker C M Association of cytotoxic T lymphocyte (CTL) escape mutations with persistent hepatitis C virus (HCV) infection. Princess Takamatsu Symp, 1995: 25: 227-235.

Yi M., Nakamoto Y., Kaneko S., Yamashita T., Murakami S. Delineation of regions important for heteromeric association of Hepatitis C virus E1 and E2. Virol. 1997a: 231: 119-129.

Zauberman, A., Nussbaum, O., Ilan, E., Eren, R., Ben-Moshe, O., Arazi, Y., Berre, S., Lubin, I., Shouval, D., Galun, E., Reisner, Y. and Dagan, S. The trimera mouse system: a mouse model for hepatitis C infection and evaluation of therapeutic agents. June 6-9, 1999; Oral 4.3. In: 6th International Symposium on Hepatitis C & Related Viruses. Bethesda USA

Zhang, L & Tam, J. P., Synthesis and application of unprotected cyclic peptides as building blocks for peptide dendrimers. J.Am. Chem. Soc., 119, 2363-2370, 1997.

Zrein, M., Louwagie, J., Boeykens, H., Govers, L., Hendrickx, G., Bosman, F., Sablon, E., Demarquilly, C., Boniface, M. and Saman, E. (1998) Assessment of a new immunoassay for serological confirmation and discrimination of human T-cell lymphotropic virus infections. Clin. Diagn. Lab. Imm. 5: 45-49.

1- An HCV protein, or any functionally equivalent part thereof, comprising at least two Cys-amino acids, which have a reversible redox status, and said Cys amino acids are comprised in the amino acid sequence Cys-X₁-X₂-Cys, in which amino acid X₁ denotes any amino acid, and amino acid X₂ denotes any amino acid.

2- The HCV protein, or any functionally equivalent part thereof, according to claim 1, in which amino acid X, denotes either amino acid Val, Leu or lie, and amino acid X₂ denotes any amino acid.

3- The HCV protein, or any functionally equivalent part thereof, according to claim 1, in which amino acid X, denotes any amino acid, and amino acid X₂ denotes amino acid Pro.

4- The HCV protein, or any functionally equivalent part thereof, according to claim 1, in which amino acid X, denotes either amino acid Val, Leu or lIe, and amino acid X₂ denotes amino acid Pro.

5- The HCV protein, or any functionally equivalent part thereof, according to claim 1, in which said HCV protein is chosen from the group E1s or E1p.

6- An HCV protein, or any functionally equivalent part thereof, comprising at least two Cys-amino acids, which have a reversible redox status, according to any of claims 1 to 5, obtainable by the following process:
(a) purifying an HCV protein, or any functionally equivalent part thereof, in which the cysteine residues are reversibly protected by chemical and/or enzymatic means,
(b) removal of the reversibly protection state of the cysteine residues,
(c) obtaining an HCV protein, or any functionally equivalent part thereof, in which the cysteine residues have a reversible redox status.

7- The HCV protein, or any functionally equivalent part thereof, according to any of claims 1 to 6 for use as a medicament.

8- Use of the HCV protein, or any functionally equivalent part thereof, according to any of claims 1 to 6 for the manufacture of an HCV vaccine composition, in particular a therapeutic vaccine composition or a prophylactic vaccine composition.

9- The HCV protein, or any functionally equivalent part thereof, according to any of claims 1 to 7, for raising antibodies, that specifically recognise said HCV protein, or any functionally equivalent part thereof.

10- Immunoassay for detecting HCV antibody, which immunoassay comprises:
(1) providing the HCV protein, or any functionally equivalent part thereof, according to any of claims 1 to 7;
(2) incubating a biological sample with said HCV protein under conditions that allow formation of HCV antibody-HCV protein complex;
(3) determining whether said HCV antibody-HCV protein complex is formed.

11- A bioassay for identifying compounds that modulate the oxido-reductase activity of HCV proteins according to any of claims 1 to 7, said bioassay comprising:
(a) exposing cells expressing HCV proteins, or any functionally equivalent part thereof, according to any of claims 1 to 7 to at least one compound whose ability to modulate the oxido-reductase activity of said proteins is sought to be determined; and thereafter
(b) monitoring said proteins for changes in oxido-reductase activity.
